(19) United States
(12) Patent Application Publication
(10) Pub. No.: US 2024/0368307 A1
(43) Pub. Date: Nov. 7, 2024

(54) METHODS AND COMPOSITIONS FOR TREATING SEPSIS

(71) Applicant: THE UNIVERSITY OF CHICAGO, Chicago, IL (US)

(72) Inventors: Nicolas CHEVRIER, Chicago, IL (US); Michihiro TAKAHAMA, Chicago, IL (US)

(73) Assignee: THE UNIVERSITY OF CHICAGO, Chicago, IL (US)

(21) Appl. No.: 18/642,451

(22) Filed: Apr. 22, 2024

Related U.S. Application Data
(60) Provisional application No. 63/461,107, filed on Apr. 21, 2023.

(51) Int. Cl. C07K 16/40; A61K 31/201; A61K 31/202; A61K 31/415; A61K 31/497; A61K 45/00; G01N 33/68

(52) U.S. Cl. CPC C07K 16/40; A61K 31/201; A61K 31/202; A61K 31/415; A61K 31/497; A61K 45/05; G01N 33/6893; C07K 2317/24; C07K 2317/565; C07K 2317/622; G01N 2333/916

(57) ABSTRACT

Aspects of the disclosure relate to methods for treating sepsis in a patient comprising administering a PLA2G5 targeting molecule, fatty acids, LPA inhibitors, and/or other compositions to the patient.

### STATEMENT OF GOVERNMENT SUPPORT

This invention was made with government support under AI1145100 awarded by the National Institutes of Health. The government has certain rights in the invention.

This application claims priority of U.S. provisional application No. 63/461,107 filed Apr. 21, 2023, which is hereby incorporated by reference in its entirety.

### BACKGROUND OF THE INVENTION

### 1. Field of the Invention

This invention relates to the field of molecular biology and medicine.

### 2. Background

Sepsis is a systemic response to infection with life-threatening consequences for the host¹. While many molecular and cellular factors have been linked to the damaging effects of sepsis on the body, knowledge of the molecular mechanisms underlying theses harmful effects remains incomplete²⁴. For example, intravascular hemolysis is a severe, well-established complication of sepsis and other disorders such as sickle cell disease, malaria infection, or beta-thalassemia⁵. In homeostatic conditions, damaged red blood cells are removed from the circulation through phagocytosis by macrophages in the spleen and liver. However, in disease conditions such as sepsis, intravascular hemolysis leads to the destruction of red blood cells and the release of free hemoglobin, heme, and iron into the circulation⁶. These degradation products lead to toxic effects for the vasculature and tissues through, for example, oxidative stress⁷ and the amplification of damaging inflammatory signals^{8,9}. In addition, the plasma levels of free hemoglobin, heme, and iron have each been linked with increased mortality in sepsis¹⁰⁻¹². However, the molecular mechanisms underlying hemolysis during sepsis remain unclear with candidate mechanisms including the coagulation and complement systems or the direct membrane effects of lipopolysaccharide¹³. As a result, suitable targets to decrease hemolysis in sepsis are lacking.

Secreted phospholipase A₂ (sPLA₂) enzymes are found in mammalian tissues and snake venom. sPLA₂ enzymes hydrolyze the glycerol backbone of phospholipids to release fatty acids and lysophospholipids and have been collectively involved in a vast array of biological functions in health and disease^{14,15} Each sPLA2 enzyme's pleiotropic functions result from both its intrinsic catalytic properties and substrate preferences and its changes in expression across cell types, tissues, and disease conditions¹⁴. For example, sPLA₂ group V (PLA2G5) has several local pathogenic and protective roles linked to various cell types, including macrophages, adipocytes, endothelial cells, bronchial epithelial cells, and cardiomyocytes¹⁶⁻²². PLA2G5 has also been shown to be induced by lipopolysaccharide (LPS) in a model of acute lung injury²³. However, the expression and function of PLA2G5 during systemic inflammation such as sepsis has not been studied in mouse or human.

### SUMMARY OF THE INVENTION

Aspects of the disclosure relate to a method for treating sepsis in a patient comprising administering a PLA2G5-targeting molecule to the patient. Further aspects of the disclosure relate to a method for treating an sepsis in a patient comprising administering a fatty acid and/or LPA inhibitor to the patient. Yet further aspects of the disclosure relate to compositions comprising a PLA2G5-targeting molecule, a fatty acid, an LPA inhibitor, and/or one or more additional therapeutic agent(s). Methods and compositions are provided relating to the treatment of sepsis involving inhibiting or reducing the amount, activity, and/or expression of PLA2G5 in a patient. Certain compositions disclosed herein are capable of reducing the amount, activity, and/or expression of PLA2G5 in a patient.

Aspects of the disclosure include various methods directed to a patient including methods for treating sepsis, reducing the severity of sepsis, improving the efficacy of a sepsis treatment, reducing the toxicity of PLA2G5, reducing lipid dysbiosis in sepsis, and reducing gut disruption in sepsis, any of which may include 1, 2, 3, 4, or more steps including any of the following: administering a PLA2G4-targeting molecule to the patient, administering a fatty acid, administering an LPA inhibitor to the patient, administering a therapy useful for treatment of sepsis (including any additional therapy) to the patient, monitoring the patient for sepsis, and taking a biological sample from the patient. In some aspects, the step is performed once. In some aspects, the step is performed as many times as necessary to effect a desired outcome, including performing the step 1, 2, 3, 4, 5, 6, 7, 8, 9, 10, or more times.

In some aspects, the patient has, is suspected of having, has been diagnosed with, or has symptoms of intravascular hemolysis. In certain aspects, the patient has, is suspected of having, or has been diagnosed with having systemic inflammatory response syndrome (SIRS). In certain aspects, the patient has, is suspected of having, or has been diagnosed with having sepsis. In certain aspects, the patient has, is suspected of having, or has been diagnosed with having severe sepsis. In certain aspects, the patient has, is suspected of having, or has been diagnosed with having septic shock and/or multiorgan dysfunction syndrome (MODS). In some aspects, the patient is a human. In some aspects, the patient is a human.

In some aspects, the patient has or has not been tested for the presence of PLA2G5, a fatty acid, or any other marker for sepsis in a biological sample from the patient. In some aspects, the patient has been previously treated for sepsis. In some aspects, the previous treatment comprises an antibiotic, fluids, insulin, a pain medication, oxygen, a blood transfusion, folic acid, corticosteroids, an immunoglobulin, rituximab, surgery, a stem cell transplant, a vasopressor, and any combination thereof.

In some aspects, the method further comprises administration of an additional therapy or the composition comprises an additional therapeutic agent. In some aspects, the patient receives an administration of an additional therapy. In some aspects, the additional therapy or agent comprises an antibiotic, fluids, insulin, a pain medication, oxygen, a blood transfusion, folic acid, corticosteroids, an immunoglobulin, rituximab, surgery, a stem cell transplant, a vasopressor, and any combination thereof.

In some aspects, the PLA2G5 targeting molecule comprises an anti-PLA2G5 antibody or a PLA2G5-binding fragment thereof. In some aspects, the antibody is humanized or chimeric. In some aspects, the antibody is conjugated to a molecule.

In some aspects, the PLA2G5 targeting molecule comprises a heavy chain variable region and/or a light chain variable region from a PLA2G5 antibody. In some aspects, the PLA2G5 targeting molecule comprises a CDR1, CDR2, and CDR3 from a heavy chain variable region and/or a CDR1, CDR2, and CDR3 from a light chain variable region. The heavy chain and light chain variable regions may be from or derived from a PLA2G5 antibody. In some aspects, the PLA2G5 targeting molecule comprises a single chain variable fragment (scFV). The scFv may comprise hypervariable regions, such as CDR1, CDR2, and CDR3 from a heavy chain variable region and/or a CDR1, CDR2, and CDR3 from a light chain variable region from an anti-PLA2G5 antibody.

Certain aspects relate to fatty acids, including LPA, oleic acid (18:1), and/or linoleic acid (18:2), 12-hydroxyeicosatetraenoic acid (12-HETE), 13-hydroxyoctadecadienoic acid (13-HODE), 9-hydroxyoctadecadienoic acid (9-HODE), 9-oxo-octadecadienoic acid (9-oxo-ODE), beraprost, oleic acid (18:1), linoleic acid (18:2), LPC, LPE, and any combination thereof. The LPA may be LPA 16:0, LPA 18:0, LPA 18:1, and any combination thereof. It is specifically contemplated that in some aspects, one or more of the fatty acids are excluded. In some aspects, the fatty acids are administered to a patient to treat sepsis.

Certain aspects relate to LPA inhibitors, such as PF8380 and/or Ki6425. The KPA inhibitors may be administered to a patient to treat sepsis.

In some aspects, the a biological sample from the patient has been determined to be positive for one or more sepsis markers. The sepsis markers may be any marker (such as a biomarker) or other diagnostically relevant symptom (including but not limited to fever, rash, confusion, and chills) that are useful in diagnosing sepsis. The markers may include, but are not limited to, blood gases, platelet count, fibrin degradation products, white blood cell count, and white blood cell differential. The biomarkers of sepsis include, but are not limited to, soluble receptors relevant to sepsis, membrane receptors relevant to sepsis, DAMP molecules, chemokine ligands relevant to sepsis, acute phase proteins relevant to sepsis, miRNAs relevant to sepsis, and non-coding RNAs relevant to sepsis. The biomarkers may include other biomarkers disclosed herein.

PLA2G5 targeting molecules, fatty acids, and/or LPA inhibitors useful in the methods and compositions of the disclosure are known in the art. It is contemplated that the PLA2G5 targeting molecules described as useful for other indications may be used in the method and composition aspects of the current disclosure. In some aspects, the PLA2G5 targeting molecule comprises a PLA2G5 CAR, which is a PLA2G5 polypeptide fused to the transmembrane region and intracellular signaling region of a CAR molecule, such as 41BB and CD3-zeta. The PLA2G5 polypeptide may be full length polypeptide or a fragment or truncated version thereof that interacts and binds to PLA2G5. The CARs of the disclosure may be expressed on T cells or NK cells.

In some aspects, the biological sample comprises a blood sample. In some aspects, the biological sample comprises a biopsy. In some aspects, the biological sample is one obtained by methods such fine needle aspiration, core needle biopsy, vacuum assisted biopsy, incisional biopsy, excisional biopsy, punch biopsy, shave biopsy or skin biopsy. In certain aspects the sample is obtained from a biopsy from lung tissue by any of the biopsy methods previously mentioned. In certain aspects of the current methods, any medical professional such as a doctor, nurse or medical technician may obtain a biological sample for testing. Yet further, the biological sample can be obtained without the assistance of a medical professional. A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen. The sample may be obtained by methods known in the art. In certain aspects, the samples are obtained by biopsy.

Also disclosed are methods of preventing hemolysis, methods of preventing hemolysis during sepsis in a patient, methods of preventing organ failure in a patient, methods of preventing organ failure during sepsis in a patient, methods of preventing organ injury, methods of preventing organ injury during sepsis in a patient, methods of preventing multi-organ injury, methods of preventing multi-organ injury during sepsis in a patient, methods of preventing multi-organ failure, and methods of preventing multi-organ failure during sepsis in a patient. In some aspects, the method comprises administering a PLA2G5 targeting molecule, a fatty acid, an LPA inhibitor, or a combination thereof, to the patient.

Also disclosed are methods of measuring protein levels in a patient. In certain aspects, the method comprises measuring a PLA2G5 gene product in a biological sample from the patient, including any patient described herein. In certain aspects, the patient has, is suspected of having, or has been diagnosed with having sepsis. In certain aspects, the PLA2G5 gene product is a PLA2G5 RNA. In certain aspects, the PLA2G5 gene product is a PLA2G5 protein. In certain aspects, the biological sample is a biological sample from the patient's colon. In certain aspects, the biological sample is a sample from the patient's small intestine. In certain aspects, the biological sample comprises goblet cells. In certain aspects, the biological sample comprises secretory cells.

Also disclosed are methods of diagnosing sepsis in a patient. In certain aspects, the method comprises measuring a PLA2G5 gene product in a biological sample from the patient. In certain aspects, the PLA2G5 gene product is a PLA2G5 RNA. In certain aspects, the PLA2G5 gene product is a PLA2G5 protein. In certain aspects, the biological sample is a biological sample from the patient's colon. In certain aspects, the biological sample is a sample from the patient's small intestine. In certain aspects, the biological sample comprises goblet cells. In certain aspects, the biological sample comprises secretory cells.

Also disclosed are methods comprising administering a administering a PLA2G5 targeting molecule, a fatty acid, an LPA inhibitor, or a combination thereof, to a patient, wherein the patient is determined to have a level of PLA2G5 gene product associated with sepsis-related mortality in a biological sample obtained from the patient. The level associated with sepsis-related mortality may be determined by a method disclosed herein. In certain aspects, the biological sample comprises a colon sample and/or plasma sample. In certain aspects, the PLA2G5 gene product is a PLA2G5 protein. In certain aspects, the PLA2G5 gene product is a PLA2G5 RNA. In certain aspects, the level of PLA2G5 gene product is associated with SIRS. In certain aspects, the level of PLA2G5 gene product is associated with sepsis. In certain aspects, the level of PLA2G5 gene product is associated with severe sepsis. In certain aspects, the level of PLA2G5 gene product is associated with sepsis induced hemolysis. In certain aspects, the level of PLA2G5 gene product is associated with septic shock and/or MODS.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that aspects described in the context of the term “comprising” may also be implemented in the context of the term “consisting of” or “consisting essentially of.”

It is specifically contemplated that any limitation discussed with respect to one aspect of the invention may apply to any other aspect of the invention. Furthermore, any composition of the invention may be used in any method of the invention, and any method of the invention may be used to produce or to utilize any composition of the invention. Aspects of an aspect set forth in the Examples are also aspects that may be implemented in the context of aspects discussed elsewhere in a different Example or elsewhere in the application, such as in the Summary of Invention, Detailed Description of the Aspects, Claims, and description of Figure Legends.

Other objects, features and advantages of the present invention will become apparent from the following detailed description. It should be understood, however, that the detailed description and the specific examples, while indicating preferred aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIGS. 1A-1D. PLA2G5 is Induced in Colon Secretory Cell Types during Sepsis. (A-B) Whole-mouse spatial transcriptomics (ST) analysis of tissue clusters generated by unsupervised clustering (A) and Pla2g5 mRNA levels (B) overlaid on a greyscale H&E staining. Shown are whole-mount sections and ST data from 5-weeks old mice injected with a sublethal dose of LPS (5 mg/kg) or left untreated as control. BF, brown fat; LN, lymph node; BM, bone marrow. (C) Average expression of Pla2g5 across all ST array spots covering indicated tissues sampled in sections from A and B. Means±SEM are shown. (D) Correlation between Pla2g5 expression and cell type deconvolution predictions across each ST array spot under the colon tissue. Shown are goblet, secretory, enteroendocrine, brush, epithelial, and crypt stem cells and enterocytes.

FIGS. 2A-2G. PLA2G5 is Dynamically Upregulated in Colon by Cytokines during Sepsis. (A) Normalized counts for Pla2g5 from indicated organs in LPS sepsis. LN, lymph node. Means±SEM are shown (n=3-4). (B) Volcano plot for Pla2g5 expression at 6 hours post-LPS injection across all 12 organs (circles) from A. (C) Normalized counts for Pla2g5 from indicated organs at 16 hours post-surgery across the three indicated severity grades (mild, moderate, severe) of cecal ligation and puncture (CLP) sepsis. LN, lymph node. Means±SEM are shown (n=5). (D-E) Normalized counts for Pla2g5 in colon (D) or indicated tissues (E) from mice injected with indicated recombinant cytokines (D), LPS with or without dexamethasone (DEX) pre-treatment (E), or left untreated as control (D-E). Means±SEM are shown (n=4-8). FDR-adjusted p-values (limma): P<0.05 (*), P<0.01 (**), P<0.001 (***), P<0.0001 (****).

FIGS. 3A-3F. PLA2G5 Leads to Multi-Organ Failure Independently of Cytokines during Sepsis. (A-C) Survival curves of mice upon LPS (A-B) or CLP (C) sepsis using PLA2G5 neutralizing or mouse IgG1 isotype control antibodies (Isotype) (A) or indicated mouse genotypes (B-C) (n=10-11). (D-E) Serum levels of indicated organ injury markers at 24 hours post-injection of LPS in indicated mouse genotypes (D) or in the presence PLA2G5 neutralizing or mouse IgG1 isotype control antibodies (Isotype) (E). Means±SEM are shown (n=4-5). (F-G) Histological analysis of kidney (PAS staining) and liver, heart, and lungs (H&E staining) at 24 hours post-sublethal LPS injection from Pla2g5^{-/-} (top) or Pla2g5^{-/-} (bottom) mice (F) or mice treated with PLA2G5 neutralizing or mouse IgG1 isotype control antibodies (G). Scale bars, 100 m. Data are representative of three independent experiments. *, P<0.05; **, P<0.01; ***, P<0.001; ****, P<0.0001 by log-rank test (A-C) or a Student's t test (D-E).

FIGS. 4A-4L. PLA2G5 Does Not Impact Lipid Metabolite Levels in Colon during Sepsis. (A) Serum levels of indicated cytokines in Pla2g5^{-/-} or Pla2g5^{-/-} mice at 24 hours after LPS injection. Data are shown as means±SEM (n=4) and representative of two independent experiments. (B-L) Lipidomics profiling in colon tissues from mice after 12 hours post-sublethal LPS injection and pre-treated with anti-Pla2g5 neutralizing antibodies or left untreated. Shown are mass spectrometry intensities for indicated lipid metabolites: FA, fatty acid; LPA, lysophosphatidic acid; LPC, lysophosphatidylcholine; LPE, lysophosphatidylethanolamine; LPG, lysophosphatidylglycerol; LPI, lysophosphatidylinositol; LPS, lysophosphatidylserine; AA, arachidonic acid; DPA, docosapentaenoic acid; EPA, eicosapentaenoic acid; DHA, docosahexaenoic acid; LA, linoleic acid, metabolites; AA, arachidonic acid; PGE2/PGD2_8-iso, PGE2/PGD2_8-iso-PGA2. Means±SEM are shown (n=4). *, P<0.05; **, P<0.01; ***, P<0.001 by Student's t test.

FIGS. 5A-5E. PLA2G5 Blockade Increases Splenic Red Pulp Macrophages and Iron Homeostasis. (A) Heatmap of differentially expressed genes (rows) from whole-tissue mRNA profiles ordered by k-means clustering (dashed lines) and organ types (top, colors) at 12 hours after sublethal LPS injection and anti-PLA2G5 antibody pre-treatment. Values are log 2 fold-changes relative to matching tissues from LPS-treated mice without antibody blockade (FDR-adjusted p-value<0.01; n=3-4). Representative enriched Gene Ontology (GO) terms within each cluster are indicated on the right (p-value<0.05). (B) Percentages (x axis) of genes differentially expressed in tissues (rows) upon pre-treatment with anti-PLA2G5 neutralizing antibodies in LPS-injected mice that match the genes regulated by LPS alone. BM, bone marrow; iLN, inguinal lymph node. (C) Heatmap of gene expression levels for genes related to splenic red pulp macrophage (rows) from whole-spleen mRNA profiles at 12 hours after sublethal LPS injection with or without anti-PLA2G5 antibody pre-treatment (columns). Values are normalized counts scaled by row (n=3). (D-E) Images of Prussian blue staining for ferric iron on spleen histological sections from LPS-injected Pla2g5^{-/-} (top) or Pla2g5^{-/-} (bottom) mice (D). Insets in D match the magnified areas shown in E. Scale bars, 2 mm (D) and 200 μm (E). Images are representative of two independent experiments.

FIGS. 6A-6F. PLA2G5 lyses erythrocyte membrane phospholipids in vitro, leading to hemolysis. (A-D) In vitro hemolysis assay. Shown are percentages of cell-free oxyhemoglobin (relative to an equal volume of chemically lysed erythrocytes) in cell lysates from normal (Control) or phosphatidylserine (PS)-exposing (Damaged) erythrocytes incubated with recombinant PLA2G5 (rPLA2G5) or BSA for 1 hour at 37° C. (A), PS-exposing erythrocytes incubated with indicated concentrations of rPLA2G5 for 1 hour at 37° C. (B), PS-exposing erythrocytes incubated with rPLA2G5 in the presence or absence of the secreted phospholipase A2 inhibitor Varespladib or left untreated (control) (C), or PS-exposing erythrocytes incubated with rPLA2G5 in isotonic, hypotonic, or hypertonic solution (D). Means±SEM are shown (n=4). Data are representative of three independent experiments. (E-F) Lipidomics profiling of normal or PS-exposing erythrocytes incubated with rPLA2G5 for 1 hour at 37° C. Shown are fold-changes relative to normal erythrocytes (E) and mass spectrometry intensities (F) for indicated lipid metabolites. LPA, lysophosphatidic acid; LPC, lysophosphatidylcholine; LPE, lysophosphatidylethanolamine; LPG, lysophosphatidylglycerol; LPI, lysophosphatidylinositol; LPS, lysophosphatidylserine; OA, oleic acid; LA, linoleic acid; AA, arachidonic acid; EPA, eicosapentaenoic acid; DHA, docosahexaenoic acid. Means±SEM are shown (n=3). *, P<0.05; **, P<0.01; ***, P<0.001; ****, P<0.0001 by one-way ANOVA with Tukey-Kramer test or Student's t test.

FIGS. 7A-7J. PLA2G5 Acts as a Self-Venom during Sepsis by Mediating Intravascular Hemolysis. (A) Plasma heme concentrations in Pla2g5^{+/+} or Pla2g5^{-/-} mice 24 hours post-sub-lethal LPS or PBS (control) injections. Means±SEM are shown (n=4). (B) Plasma concentration of oxyhemoglobin and heme in mice 12 hours post-lethal LPS injection and pre-treated with anti-PLA2G5 neutralizing antibodies or isotype control. Means±SEM are shown (n=5). (C-D) Plasma concentration of oxyhemoglobin and heme (C) and serum levels of BUN and ALT (D) in mice injected with recombinant PLA2G5 (rPLA2G5) or saline only (Control). Means±SEM are shown (n=4-5). (E-F) Plasma oxyhemoglobin levels in mice injected with plasma which were collected from LPS- or PBS (Control)-injected mice and incubated in vitro with anti-PLA2G5 neutralizing antibodies for 4 hours at 4° C. prior to transfer into naive mice. Means±SEM are shown (n=3). (G-H) Plasma oxyhemoglobin levels in mice injected with plasma collected from LPS-injected Pla2g5^{-/-} or Pla2g5^{-/-} mice or PBS-injected Pla2g5^{-/-} mice as control. Means±SEM are shown (n=4). (I) Box plots depicting Log 2 relative fluorescence units of PLA2G5 level in plasma of patients with COVID-19 by severity over time (Methods). Patients were classified by acuity levels based on the World Health Organization (WHO) Ordinal Outcomes Scale. Acuity levels were determined by their severity condition within 28 days post-enrollment as follows: A1, death within 28 days; A2, intubation, mechanical ventilation; A3, hospitalized and requiring supplemental oxygen; A4, hospitalized without requiring supplemental oxygen; and A5, discharged directly from the Emergency Department without subsequently returning and requiring admission within 28 days. Median and InterQuartile Range (IQR) of Sequential Organ Failure Assessment (SOFA) score for each acuity levels are 8 and 5-10 (A1); 7 and 6-9 (A2); 1 and 0-3 (A3); 0 and 0-1 (A4); 1 and 0-1 (A5). A1 and A2 were classified as sepsis (dark grey) and A3-A5 as non-sepsis (light grey). (J) Distribution of accuracy values from a decision tree classifier analysis using PLA2G5 plasma levels at day 0 from sepsis and non-sepsis patient groups (data from panel H). Data are representative of two to three independent experiments (A-D, F, and H). *, P<0.05; **, P<0.01; ***, P<0.001 by one-way ANOVA with Tukey-Kramer test or Student's t test.

FIG. 8. Schematic depicting the proposed model for the role of PLA2G5 in sepsis. PLA2G5 causes intravascular hemolysis through its lipolytic activity on select phospholipids of erythrocyte membranes. Damaged erythrocytes are captured by splenic macrophages, leading to iron accumulation in spleen, and the degradation products from erythrocytes, such as heme, lead to toxic effects for tissues.

FIGS. 9A-9B. Spatial Transcriptomics Analysis of Pla2g5 Expression in the Gut. (A) Whole-mouse spatial transcriptomics (ST) data (FIG. 1A-B) from control and LPS conditions (columns) were magnified to only show colon (brown) and small intestine (SI, yellow) tissues (top) and Pla2g5 normalized expression (bottom) overlaid on a greyscale H&E image. (B) Cell type deconvolution analysis of each ST spot data for indicated colon cell types (top) across control and LPS conditions (rows).

FIGS. 10A-10B. Pla2g5 Expression in Public Single-Cell RNA-seq Data across Mouse Tissues. (A-B) Single-cell expression of Pla2g5 mRNAs across mouse tissues from a public database. Shown are uniform manifold approximation and projection (UMAP) plots of Pla2g5 mRNA counts (right panels; A-B) across indicated mouse organs (left panel; A) or large intestine cell types (left panel; B). BAT, brown adipose tissue; GAT, gonadal adipose tissue; MAT, mesenteric adipose tissue; SCAT, subcutaneous adipose tissue.

FIG. 11. Transcriptional Regulation of Pla2g5 across Tissues. Basal whole-tissue gene expression of Pla2g5 mRNAs across 15 tissues. Shown are normalized counts for Pla2g5 from indicated organs in naive mice. LN, lymph node; AT, adipose tissue; SI, small intestine; BM, bone marrow. Data are shown as means±SEM (n=4).

FIGS. 12A-12C. PLA2G5 Perturbation Prevents Body Temperature Loss in LPS and CLP sepsis. (A-C) Measurements of rectal temperature in mice upon LPS (A-B) or cecal ligation and puncture (CLP, C) sepsis using anti-PLA2G5 neutralizing antibodies (A) or indicated mouse genotypes (B-C). Data are representative of three independent experiments.

FIG. 13. Impact of PLA2G5 on the Plasma Levels of Glucose, Triglyceride, and Cholesterol during Sepsis. Serum levels of indicated metabolites before (untreated) and 24 hours post-injection with a sublethal LPS dose in the presence or absence of Pla2g5 blockade. Data are representative of two independent experiments and shown as means±SEM (n=4). *, P<0.05; **, P<0.01; ***, P<0.001; and ****, P<0.0001 by one-way ANOVA with Tukey-Kramer test.

FIGS. 14A-14C. Blockade of PLA2G5 Impacts Multi-Tissue Gene Expression during Sepsis. (A) Heatmap of differentially expressed genes (rows) from whole-tissue mRNA profiles ordered by k-means clustering and organ types (top, colors) at 12 hours after sublethal LPS injection (left). Values are log 2 fold-changes relative to matching organs from untreated controls for wild-type (FDR-adjusted p-value<0.01, absolute fold change >2; n=4). Genes up- or down-regulated in at least of one of the 7 tissues at 12 hours post-sublethal LPS injection in the presence of anti-PLA2G5 neutralizing antibodies (FDR-adjusted p-value<0.01; n=3-4) are indicated in black (right). (B) Heatmap of gene expression levels for indicated cell-type markers and sepsis biomarker genes (rows) from mRNA profiles at 12 hours after sublethal LPS injection in the presence (light grey) or absence (dark grey) of anti-PLA2G5 neutralizing antibodies. Values are normalized counts scaled by row. All genes shown were significantly downregulated by PLA2G5 blockade in LPS-treated mice (FDR-adjusted p-value<0.01; n=3-4). (C) Bar plots of normalized counts for indicated genes and organs from panel B. Data are shown as means±SEM (n=3-4).

### DESCRIPTION OF ILLUSTRATIVE ASPECTS

Sepsis is a systemic response to infection with life-threatening consequences such as hemolysis, a predictor of mortality risks for the disease. By measuring organism-wide changes in gene expression, aspects herein show that the secreted phospholipase PLA2G5 is induced in colon cell types during sepsis. The genetic deletion and antibody blockade of PLA2G5 abrogated the lethal effects of sepsis. PLA2G5 blockade during sepsis led to an increase in splenic red pulp macrophages and iron homeostasis, linking PLA2G5 to red blood cell homeostasis during sepsis. Mechanistically, bloodborne PLA2G5 led to intravascular hemolysis through its lipolytic activity on red blood cell membranes. In humans with sepsis, the plasma level of PLA2G5 was elevated and predictive of disease severity and mortality. Sepsis corrupts PLA2G5 from the gut into becoming a systemic self-venom which is toxic for host red blood cells.

Sepsis also causes changes in lipid profiles. Such changes can be exploited to treat inflammation and sepsis in a patient. For example, certain lipids, such as LPA, inhibit inflammation in sepsis. Administration of such lipids may be used to treat the inflammation and/or sepsis. Such lipids can mimic some of the effects of dexamethasone, a commonly used anti-inflammatory drug.

### I. Definitions

The terms “protein,” “polypeptide,” and “peptide” are used interchangeably herein when referring to a gene product.

“Homology,” or “identity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules share sequence identity at that position. A degree of identity between sequences is a function of the number of matching or homologous positions shared by the sequences. An “unrelated” or “non-homologous” sequence shares less than 60% identity, less than 50% identity, less than 40% identity, less than 30% identity, or less than 25% identity, with one of the sequences of the current disclosure.

The terms “amino portion,” “N-terminus,” “amino terminus,” and the like as used herein are used to refer to order of the regions of the polypeptide. Furthermore, when something is N-terminal to a region it is not necessarily at the terminus (or end) of the entire polypeptide, but just at the N-terminus of the region or domain. Similarly, the terms “carboxy portion,” “C-terminus,” “carboxy terminus,” and the like as used herein is used to refer to order of the regions of the polypeptide, and when something is C-terminal to a region it is not necessarily at the terminus (or end) of the entire polypeptide, but just at the C-terminus of the region or domain.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this invention that is a polynucleotide encompasses both the double-stranded form and each of two complementary single-stranded forms known or predicted to make up the double-stranded form.

Cells or a culture of cells are “substantially free” of certain reagents or elements, such as serum, signaling inhibitors, animal components or feeder cells, exogenous genetic elements or vector elements, as used herein, when they have less than 10% of the element(s), and are “essentially free” of certain reagents or elements when they have less than 1% of the element(s). However, even more desirable are cell populations wherein less than 0.5% or less than 0.1% of the total cell population comprise exogenous genetic elements or vector elements.

Cells or a culture of cells are “essentially free” of certain reagents or elements, such as serum, signaling inhibitors, animal components or feeder cells, when the culture, matrix or medium respectively have a level of these reagents lower than a detectable level using conventional detection methods known to a person of ordinary skill in the art or these agents have not been extrinsically added to the culture, matrix or medium. The serum-free medium may be essentially free of serum.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally-occurring cells or artificially modified cells (e.g., fusion cells, genetically modified cells, etc.).

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “antibody” includes monoclonal antibodies, polyclonal antibodies, dimers, multimers, multispecific antibodies and antibody fragments that may be human, mouse, humanized, chimeric, or derived from another species. A “monoclonal antibody” is an antibody obtained from a population of substantially homogeneous antibodies that is being directed against a specific antigenic site.

“Antibody or functional fragment thereof means an immunoglobulin molecule that specifically binds to, or is immunologically reactive with a particular antigen or epitope, and includes both polyclonal and monoclonal antibodies. The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The antibody may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The term functional antibody fragment includes antigen binding fragments of antibodies, including e.g., Fab′, F(ab′)₂, Fab, Fv, rIgG, and scFv fragments. The term scFv refers to a single chain Fv antibody in which the variable domains of the heavy chain and of the light chain of a traditional two chain antibody have been joined to form one chain. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment retains the ability to bind its cognate antigen at comparable affinity to the full antibody.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain aspects, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this disclosure. In contrast to polyclonal antibody preparations, which typically include several different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The phrases “pharmaceutical composition” or “pharmacologically acceptable composition” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, and Ringer's dextrose), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition may be adjusted according to well-known parameters.

The term “unit dose” or “dosage” refers to physically discrete units suitable for use in a subject, each unit containing a predetermined quantity of the therapeutic composition calculated to produce the desired responses discussed herein in association with its administration, i.e., the appropriate route and treatment regimen. The quantity to be administered, both according to number of treatments and unit dose, depends on the effect desired. The actual dosage amount of a composition of the present aspects administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any particular dose derivable therein. In non-limiting examples of a range derivable from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The use of a single chain variable fragment (scFv) is of particular interest. scFvs are recombinant molecules in which the variable regions of light and heavy immunoglobulin chains encoding antigen-binding domains are engineered into a single polypeptide. Generally, the V_{H} and V_{L} sequences are joined by a linker sequence. See, for example, Ahmad (2012) Clinical and Developmental Immunology Article ID 980250, herein specifically incorporated by reference. Described herein are BCMA-specific scFv molecules that comprise the variable regions of light and heavy immunoglobulin chains encoding BCMA-binding domains that are engineered into a single polypeptide. Similarly, the CS1-specific scFv molecules described herein comprise the variable regions of light and heavy immunoglobulin chains encoding CS1-binding domains that are engineered into a single polypeptide.

As used herein, the term “binding affinity” refers to the equilibrium constant for the reversible binding of two agents and is expressed as a dissociation constant (Kd). Binding affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more (or any derivable range therein), than the binding affinity of an antibody for unrelated amino acid sequences. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

Subject” and “patient” refer to either a human or non-human, such as primates, mammals, and vertebrates. In particular aspects, the subject is a human.

Throughout this application, the term “about” is used to indicate that a value includes the inherent variation of error for the device, the method being employed to determine the value, or the variation that exists among the study subjects.

### II. PLA2G5 Targeting Agents

### A. Antibodies

Aspects of the disclosure relate to PLA2G5 targeting agents. In some aspects, the PLA2G5 targeting agent comprises an anti-PLA2G5 antibody or a fragment thereof. In certain aspects, the PLA2G5 antibody comprises a MCL-3G1, MCL-2A5, or MCL-1B7 antibody, including those described in Munoz, et al. Hybridoma, Vol. 19(2), p. 171-176 (2000), which is incorporated by reference herein in its entirety. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable of binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986 See, e.g., Epitope Mapping Protocols, supra. Additionally, antigenic regions of proteins can also be predicted and identified using standard antigenicity and hydropathy plots.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al. Front Immunol. 2013; 4: 302; 2013)

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some aspects, bispecific antibodies can be biparatopic, wherein a bispecific antibody may specifically recognize a different epitope from the same antigen. In some aspects, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some aspects, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for aspects of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some aspects, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### 1. Functional Antibody Fragments and Antigen-Binding Fragments

a. Antigen-Binding Fragments

Certain aspects relate to antibody fragments, such as antibody fragments that bind to and/or neutralize inflammatory mediators. The term functional antibody fragment includes antigen-binding fragments of an antibody that retain the ability to specifically bind to an antigen. These fragments are constituted of various arrangements of the variable region heavy chain (VH) and/or light chain (VL); and in some aspects, include constant region heavy chain 1 (CH1) and light chain (CL). In some aspects, they lack the Fc region constituted of heavy chain 2 (CH2) and 3 (CH3) domains. Aspects of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

b. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

c. Polypeptides with Antibody CDRs & Scaffolding Domains that Display the CDRs

Antigen-binding peptide scaffolds, such as complementarity-determining regions (CDRs), are used to generate protein-binding molecules in accordance with the aspects. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### B. Chimeric Antigen Receptor

In some aspects, the PLA2G5 targeting agent comprises a PLA2G5-specific CAR molecule or cells, such as T cells comprising and/or expressing a PLA2G5-specific CAR molecule. Chimeric antigen receptor T cells, or CAR T cells, are T cells from either patient, donor, or produced in vitro, that are genetically modified to express chimeric receptors specific to a sepsis antigen, along with a signaling domain and co-stimulatory molecules. This fusion of the antibody-derived single chain variable fragment with the T cell intracellular signaling domains endows the CAR T cell with the ability to recognize the sepsis antigen in a non-MHC-restricted manner.

A CAR molecule typically comprises one or more antibody binding region(s), an extracellular spacer, a transmembrane domain, and a cytoplasmic region. These are further described below.

### 1. Antigen Binding Regions

The antigen-binding region may be a single-chain variable fragment (scFv) derived from a PLA2G5 antibody. “Single-chain Fv” or “scFv” antibody fragments comprise the VH and VL domains of an antibody, wherein these domains are present in a single polypeptide chain. In some aspects, the antigen-binding domain further comprises a peptide linker between the VH and VL domains, which may facilitate the scFv forming the desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T-cell receptors, generated by B cells and T cells respectively, where these molecules bind to their specific antigen. Since most sequence variation associated with immunoglobulins and T-cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

### 2. Extracellular Spacer

An extracellular spacer may link the antigen-binding domain to the transmembrane domain. It should be flexible enough to allow the antigen-binding domain to orient in different directions to facilitate antigen binding. In one aspect, the spacer is the hinge region from IgG. Alternatives include the CH2CH3 region of immunoglobulin and portions of CD3.

As used herein, the term “hinge” refers to a flexible polypeptide connector region (also referred to herein as “hinge region” or “spacer”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1 (Burton (1985) Molec. Immunol., 22: 161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

### 3. Transmembrane Domain

The transmembrane domain is a hydrophobic alpha helix that spans the membrane. Different transmembrane domains may result in different receptor stability.

The transmembrane domain is interposed between the extracellular spacer and the cytoplasmic region. In some aspects, the transmembrane domain is interposed between the extracellular spacer and one or more costimulatory regions. In some aspects, a linker is between the transmembrane domain and the one or more costimulatory regions. In some aspects, the transmembrane domain is derived from CD28, CD8, CD4, CD3-zeta, CD134, or CD7.

### 4. Cytoplasmic Region

After antigen recognition, receptors cluster and a signal is transmitted to the cell through the cytoplasmic region. In some aspects, the costimulatory domains described herein are part of the cytoplasmic region.

Cytoplasmic regions and/or costimulatiory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some aspects, the cytoplasmic region includes at least one (e.g., one, two, three, four, five, six, etc.) ITAM motif as described herein. In some aspects, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX₁X₂(L/I), where X₁ and X₂ are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX₁X₂(L/I))(X₃)ₙ(YX₁X₂(L/I)), where n is an integer from 6 to 8, and each of the 6-8 X₃ can be any amino acid.

A suitable cytoplasmic region may be an ITAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM.

### III. Cells

Certain aspects relate to cells comprising polypeptides or nucleic acids of the disclosure, such as PLA2G5-targeting agents. In some aspects the cell is an immune cell or a T cell. “T cell” includes all types of immune cells expressing CD3 including T-helper cells, cytotoxic T-cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell or progenitor cell obtained from an individual. In some aspects, the cell used in therapy of a patient is autologous. In some aspects, the cell used in therapy of a patient is non-autologous.

### IV. Methods of Treatment

Aspects of the current disclosure relate to methods for treating sepsis and/or hemolytic anemia. In certain aspects, the hemolytic anemia is intravascular hemolysis. The sepsis may be at a certain stage of sepsis. The sepsis may be at any stage of sepsis.

Certain aspects, relate to methods of treating systemic inflammatory response syndrome (SIRS), methods of treating sepsis, methods of treating severe sepsis, and methods of treating septic shock and/or multiorgan dysfunction syndrome (MODS).

The methods generally involve administering a PLA2G5 targeting molecule, a fatty acid, and/or an LPA inhibitor to a patient. In some aspects, the PLA2G5 targeting molecule is a genetically modified mammalian cell with an expression vector, or an RNA (e.g., in vitro transcribed RNA), comprising nucleotide sequences encoding a polypeptide that target PLA2G5. The cell can be an immune cell (e.g., a T lymphocyte or NK cell), a stem cell, a progenitor cell, etc. In some aspects, the cell is a cell described herein or the progeny thereof.

Aspects of the disclosure include ex vivo methods. For example, a T lymphocyte, a stem cell, or an NK cell (or cell described herein) is obtained from an individual; and the cell obtained from the individual is genetically modified to express a PLA2G5 targeting molecule of the disclosure. In some cases, the genetically modified cell is activated ex vivo. In other cases, the genetically modified cell is introduced into an individual (e.g., the individual from whom the cell was obtained); and the genetically modified cell is activated in vivo.

In some aspects, the methods relate to administration of the cells or PLA2G5 targeting molecules for the treatment of a sepsis or administration to a person with a sepsis. Such methods include methods of treating a disease, including sepsis and/or hemolytic anemia.

### V. Administration of Therapeutic Compositions

The therapy provided herein may comprise the administration of one or a combination of therapeutic agents, such as one or a combination of targeting molecules, (including any targeting molecule disclosed herein), a fatty acid (including any fatty acid disclosed herein) an LPA inhibitor (including any LPA inhibitor disclosed herein) and/or other therapeutic compositions (including those useful for treating disorders disclosed herein, such as any blood disorder) to a patient. In some aspects, the therapy is a cocktail of targeting molecules, fatty acid, and/or LPA inhibitor. In some aspects, the other therapeutic compositions are useful for reducing symptoms of sepsis or other diseases disclosed herein and/or reducing side effects of the other therapeutic agents administered. The therapies may be administered in any suitable manner known in the art. In some aspects, a first therapeutic composition (such as a targeting molecule) and a second composition (such as another targeting molecule, an LPA inhibitor, or another therapeutic composition) may be administered sequentially (at different times) or concurrently (at the same time). In some aspects, the first and second therapeutic compositions are administered in a separate composition. In some aspects, the first and second therapeutic compositions are in the same composition.

In some aspects, the first therapeutic composition and the second therapeutic composition are administered substantially simultaneously. In some aspects, the first therapeutic composition and the second therapeutic composition are administered sequentially. In some aspects, the first therapeutic composition, the second therapeutic composition, and a third therapeutic composition are administered sequentially. In some aspects, the first therapeutic composition is administered before administering the second therapeutic composition. In some aspects, the first therapeutic composition is administered after administering the second therapeutic composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the therapy is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, a single dose of the targeting molecule, fatty acid, LPA inhibitor, or other therapeutic composition is administered. In some aspects, multiple doses of the targeting molecule, fatty acid, LPA inhibitor, or other therapeutic composition are administered. In some aspects, the targeting molecule, fatty acid, LPA inhibitor, or other therapeutic composition is administered at a dose of between 1 mg/kg and 5000 mg/kg. In some aspects, the targeting molecule, fatty acid, LPA inhibitor, or other therapeutic composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range derivable therein.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 day, week, month, or year intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### B. Pharmaceutical Compositions

In certain aspects, the compositions or agents, including those for use in the methods disclosed herein, such as one or more targeting molecules, fatty acids, and/or LPA inhibitors are suitably contained in a pharmaceutically acceptable carrier. The carrier can be non-toxic, biocompatible, and selected so as not to detrimentally affect the biological activity of the agent. The agents in some aspects of the disclosure may be formulated into preparations for local delivery (i.e. to a specific location of the body, such as the brain, nervous tissue, or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### C. Proteins

The nucleotides as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

### D. Other Agents

It is contemplated that other agents may be used in combination with certain aspects of the present aspects to improve the therapeutic efficacy of treatment. These additional agents include agents that act in combination and/or synergistically with the ASOs described herein. The additional agents may comprise agents that reduce symptoms of the disorders disclosed herein, or may comprise agents that reduce side effects associated with the therapeutic compositions disclosed herein.

### VI. Pharmaceutical Compositions

The present disclosure includes methods for treating disease in a subject in need thereof. The disclosure includes proteins, fatty acids, small molecules, and/or cells that may be in the form of a pharmaceutical composition that can be used to treat sepsis.

Administration of the compositions according to the current disclosure will typically be via any common route. This includes, but is not limited to parenteral, orthotopic, intradermal, subcutaneous, intramuscular, intraperitoneal, or intravenous injection.

Typically, compositions of the disclosure are administered in a manner compatible with the dosage formulation, and in such amount as will be therapeutically effective. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals. The course of the administrations may be followed by assays for alloreactive immune responses and T cell activity.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

The pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (i.e. cells of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

Methods of the disclosure include administration of a combination of therapeutic agents and/or administration of therapeutic agents, such as fecal matter and therapeutic regimens, such as steroid therapy or anti-integrin therapy, for example. The therapy may be administered in any suitable manner known in the art. For example, the therapies may be administered sequentially (at different times) or concurrently (at the same time). In some aspects, the therapies are in a separate composition. In some aspects, the therapies are in the same composition.

Various combinations of the therapies may be employed, for example, one therapy designated “A” (such as a PLA2G5-targeting molecule) and another therapy designated “B” (such as a fatty acid, LPA inhibitor, or other therapy used to treat sepsis):
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

The therapies of the disclosure may be administered by the same route of administration or by different routes of administration. In some aspects, the therapy is administered intracolonically, intravenously, intramuscularly, subcutaneously, topically, orally, transdermally, intraperitoneally, intraorbitally, by implantation, by inhalation, intrathecally, intraventricularly, or intranasally. In some aspects, the microbial modulator is administered intravenously, intramuscularly, subcutaneously, topically, orally, transdermally, intraperitoneally, intraorbitally, by implantation, by inhalation, intrathecally, intraventricularly, or intranasally.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some aspects, the therapeutically effective or sufficient amount of a therapeutic composition that is administered to a human will be in the range of about 0.01 to about 50 mg/kg of patient body weight whether by one or more administrations. In some aspects, the therapeutic agent used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some aspects, the therapeutic agent is administered at 15 mg/kg. However, other dosage regimens may be useful. In one aspect, a therapeutic agent described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VII. Examples

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### A. Example 1: Introduction

Sepsis is a systemic response to infection with life-threatening consequences for the host¹. While many molecular and cellular factors have been linked to the damaging effects of sepsis on the body, knowledge of the molecular mechanisms underlying theses harmful effects remains incomplete²⁴. For example, intravascular hemolysis is a severe, well-established complication of sepsis and other disorders such as sickle cell disease, malaria infection, or beta-thalassemia⁵. In homeostatic conditions, damaged red blood cells are removed from the circulation through phagocytosis by macrophages in the spleen and liver. However, in disease conditions such as sepsis, intravascular hemolysis leads to the destruction of red blood cells and the release of free hemoglobin, heme, and iron into the circulation⁶. These degradation products lead to toxic effects for the vasculature and tissues through, for example, oxidative stress⁷ and the amplification of damaging inflammatory signals^{8,9}. In addition, the plasma levels of free hemoglobin, heme, and iron have each been linked with increased mortality in sepsis¹⁰⁻¹². However, the molecular mechanisms underlying hemolysis during sepsis remain unclear with candidate mechanisms including the coagulation and complement systems or the direct membrane effects of lipopolysaccharide¹³. As a result, the inventors lack suitable targets to decrease hemolysis in sepsis.

Secreted phospholipase A₂ (sPLA₂) enzymes are found in mammalian tissues and snake venom. sPLA₂ enzymes hydrolyze the glycerol backbone of phospholipids to release fatty acids and lysophospholipids and have been collectively involved in a vast array of biological functions in health and disease^{14,15}. Each sPLA2 enzyme's pleiotropic functions result from both its intrinsic catalytic properties and substrate preferences and its changes in expression across cell types, tissues, and disease conditions¹⁴. For example, sPLA₂ group V (PLA2G5) has several local pathogenic and protective roles linked to various cell types, including macrophages, adipocytes, endothelial cells, bronchial epithelial cells, and cardiomyocytes¹⁶⁻²². PLA2G5 has also been shown to be induced by lipopolysaccharide (LPS) in a model of acute lung injury²³. However, the expression and function of PLA2G5 during systemic inflammation such as sepsis has not been studied in mouse or human.

In aspects herein, the inventors discovered that PLA2G5 is induced in gut cells during sepsis by using organism-wide, spatiotemporal gene expression profiling in mouse models of sepsis. The inventors found that mice treated with anti-PLA2G5 neutralizing antibodies or lacking the Pla2g5 gene were protected from lethal sepsis. While PLA2G5 blockade did not impact the levels of inflammatory cytokines or lipid metabolites, it led to a striking increase in splenic red pulp macrophages and iron homeostasis. Mechanistically, the inventors found that the lipolytic activity of PLA2G5 led to intravascular hemolysis and multi-tissue injury through the degradation products of red blood cells and cell-free hemoglobin, such as heme. Moreover, in humans with viral sepsis, the inventors found that high plasma levels of PLA2G5 were associated with increased mortality risks.

### B. Example 2: PLA2G5 is Induced in the Gut in Mouse Models of Sepsis

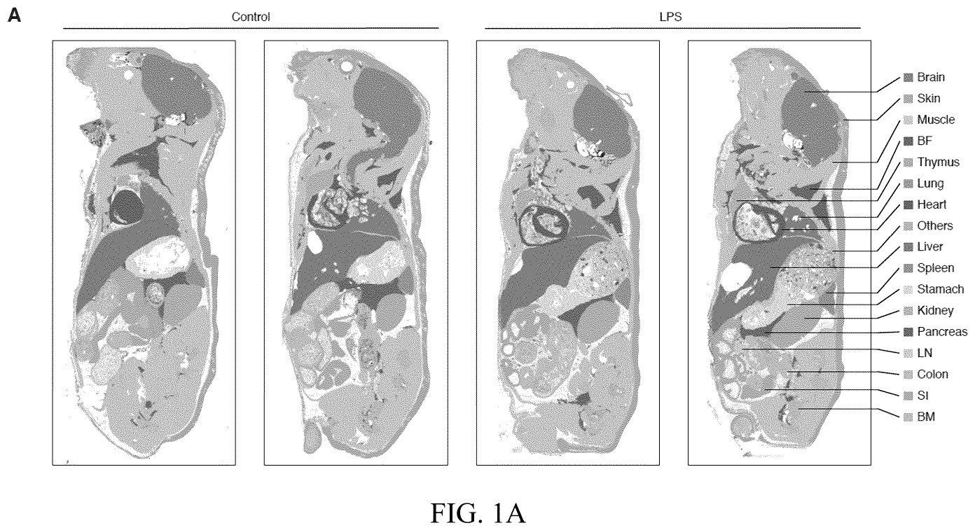

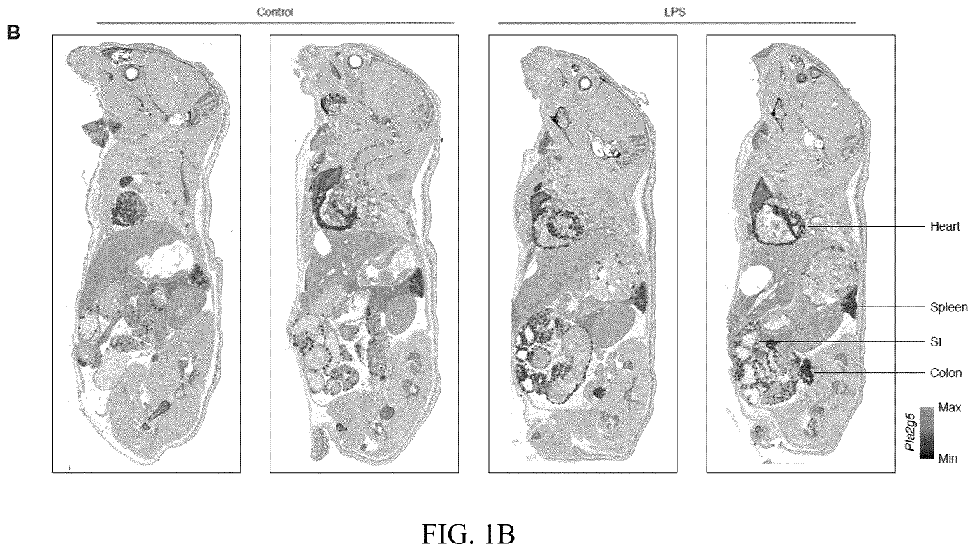

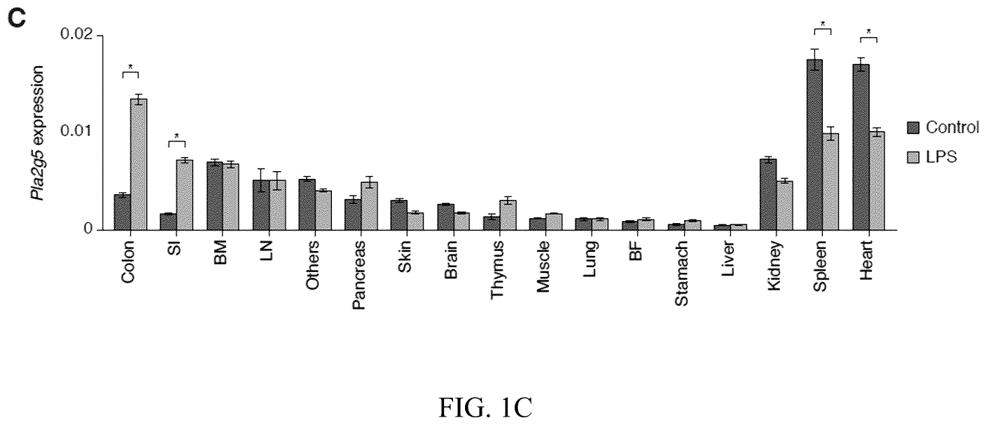

The inventors first tested if PLA2G5 was transcriptionally regulated across the body during sepsis. Using the intra-peritoneal injection of a sublethal dose (5 mg/kg) of lipopolysaccharide (LPS) to mimic systemic bacterial inflammation²⁴, the inventors measured changes in Pla2g5 gene expression across whole-mount, sagittal mouse sections using a custom, large-format spatial transcriptomics method. Out of the 17 tissue types profiled using whole mouse sections from animals injected with LPS or left untreated as control, the inventors found Pla2g5 transcripts to be significantly upregulated in the colon and small intestine and downregulated in the heart and spleen at 12 hours post-LPS injection (FIGS. 1A-C and FIG. S1A). To identify the cell types in which Pla2g5 mRNAs were upregulated in the gut, the inventors used a deconvolution method²⁵ with publicly available mouse tissue single-cell RNA-seq data as a reference to estimate the cell-type composition for each spot of the arrays used for spatial transcriptomics profiling. The analysis identified colon goblet cells and secretory cells as the cell types in which the expression of the Pla2g5 gene is upregulated during sepsis (FIG. 1D and FIG. S1B). In addition, the inventors found that the Pla2g5 gene was expressed at steady state in colon goblet cells and secretory cells (FIG. S2A-B), albeit at lower levels than in LPS sepsis.

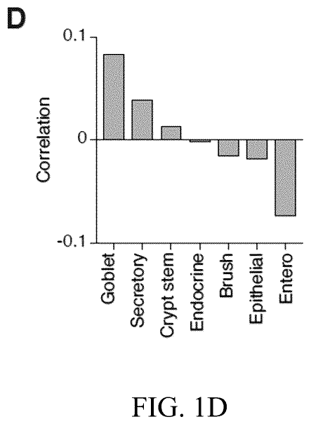

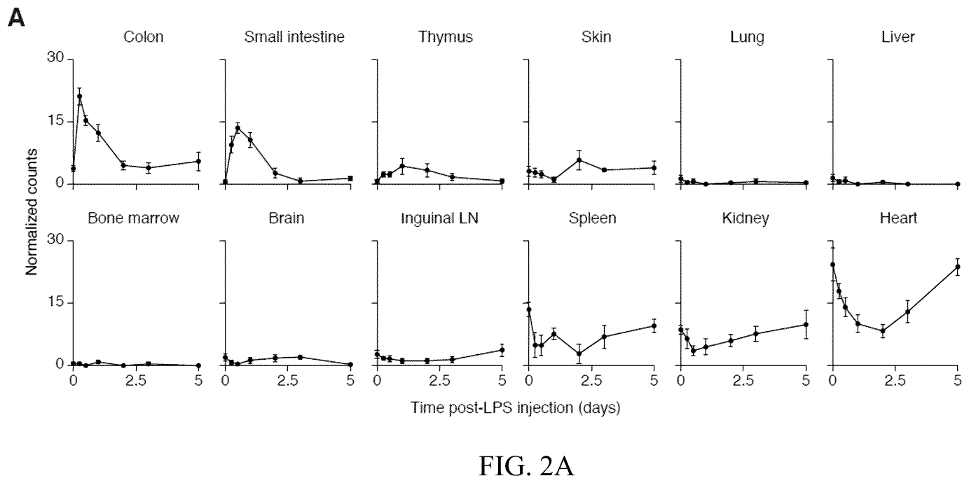

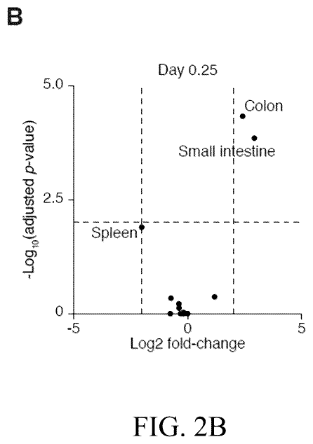

The inventors next investigated the temporal changes in Pla2g5 gene expression in the LPS sepsis model using whole-tissue mRNA profiling at 0.25, 0.5, 1, 2, 3, and 5 days post-sublethal LPS injection. Pla2g5 was strongly upregulated in the colon and small intestine within 6 to 12 hours post-LPS injection and returned to baseline within 2 to 3 days (FIG. 2A-B). In addition, heart and spleen Pla2g5 mRNA levels were downregulated upon LPS injection and returned to control levels by day 5 post-sepsis induction (FIG. 2A). In steady state conditions, heart and spleen were the two tissues with the highest levels of Pla2g5 gene expression out of 17 tissue types profiled using both whole-mouse spatial transcriptomics (FIG. 1C) and whole-tissue RNA-seq (FIG. 2A and FIG. S3A).

Next, the inventors sought to test if Pla2g5 gene expression in tissues was impacted after cecal ligation and puncture (CLP), which is considered the gold standard model for sepsis due to its high clinical relevance^{27,28}. The inventors used CLP to trigger a polymicrobial infection starting in the abdominal cavity and ranging in disease severity from mild to severe based on the position of the cecal ligation and the number and size of cecal punctures (Methods)²⁷. The inventors found that Pla2g5 mRNA levels were upregulated in the colon in all three CLP severity grades (FIG. 2C).

Lastly, the inventors asked what molecular factors regulate Pla2g5 gene expression in the colon. The inventors tested the effects of 6 recombinant cytokines (IFN-γ, IL-1β, IL-10, IL-6, IL-18, TNF) key in sepsis by injecting them intravenously alone or in all 15 pairwise combinations and measuring colon Pla2g5 expression. The inventors found that Pla2g5 gene expression was significantly upregulated in colon upon injection of recombinant TNF plus IFN-7 and IL-18 (FDR<0.05), but not other cytokine singles and pairs tested (FIG. 2D). The inventors used a corticosteroid drug, dexamethasone, with known anti-inflammatory effects to decrease inflammatory cytokine production during sepsis and found that it abrogated Pla2g5 gene induction during sepsis in the gut, but not in heart, suggesting the existence of different signals regulating Pla2g5 across tissue during sepsis (FIG. 2E). Taken together, the spatiotemporal analysis of gene expression uncovered that Pla2g5 is induced in the gut during sepsis by specific inflammatory cytokines.

### C. Example 3: PLA2G5 is Harmful for the Host During Sepsis

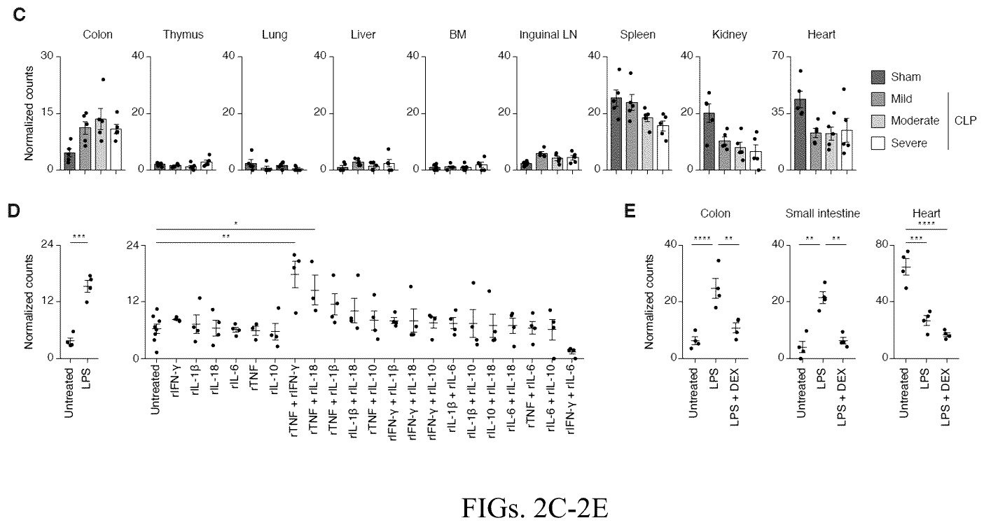

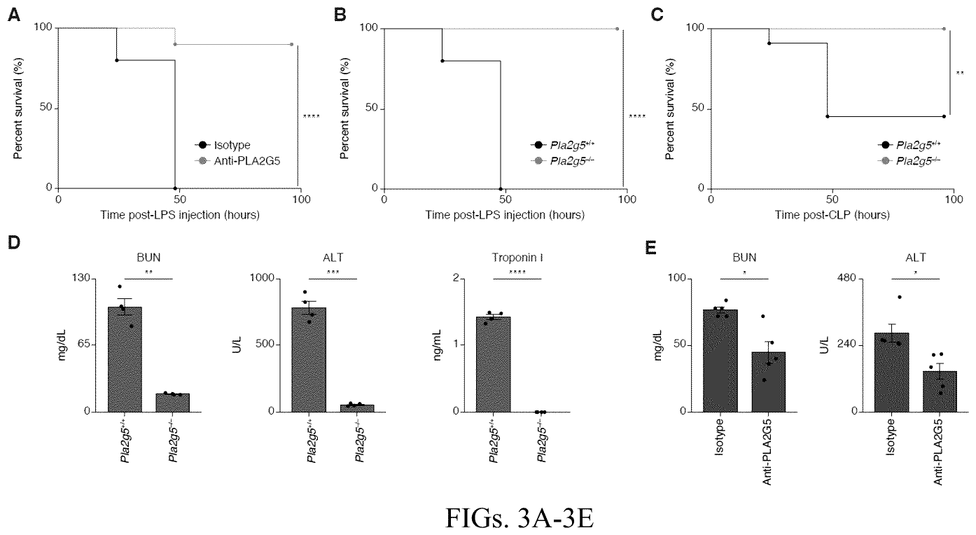

To test if PLA2G5 plays a role in the pathogenesis of sepsis, the inventors injected mice with PLA2G5-neutralizing or isotype control antibodies 1 hour prior to a challenge with a lethal dose (15 mg/kg) of LPS. The inventors found that neutralization of PLA2G5 with antibodies²⁹ significantly increased survival and prevented body temperature loss upon lethal LPS challenge (FIG. 3A and FIG. S4A). To further investigate the detrimental role of PLA2G5 in sepsis, the inventors used mice carrying a genetic deletion for the Pla2g5 gene³⁰. The inventors found that Pla2g5^{-/-} mice did not exhibit a significant loss in body temperature and survived both lethal LPS and CLP sepsis challenges whereas their wild-type counterparts did not (FIGS. 3B-C and FIG. S4B-C).

To investigate how PLA2G5 promotes the lethal effects of sepsis, the inventors first measured tissue injury markers and found that both genetic deletion and neutralization of PLA2G5 led to a decrease in the serum levels of renal, liver, and cardiac injury markers—blood urea nitrogen (BUN), alanine aminotransferase (ALT), and troponin I, respectively—during sepsis (FIG. 3D-E). The inventors did not observe differences in other systemic metabolic parameters such as blood glucose, triglyceride, and cholesterol levels upon PLA2G5 blockade during sepsis (FIG. S5A). Furthermore, both genetic deletion and neutralization of PLA2G5 led to decreased signs of histopathology in tissue sections during sepsis (FIG. 3F-G). Thus, the results suggested that PLA2G5 regulates tissue damage and survival during LPS and CLP sepsis.

### D. Example 4: PLA2G5 does not Impact Systemic Cytokine Signaling or Colon Lipid Metabolites in Sepsis

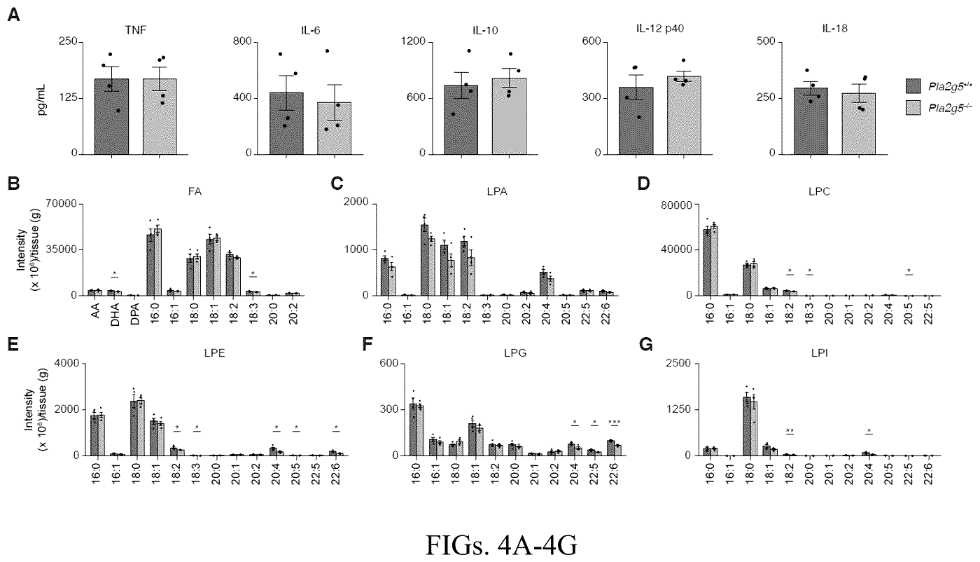

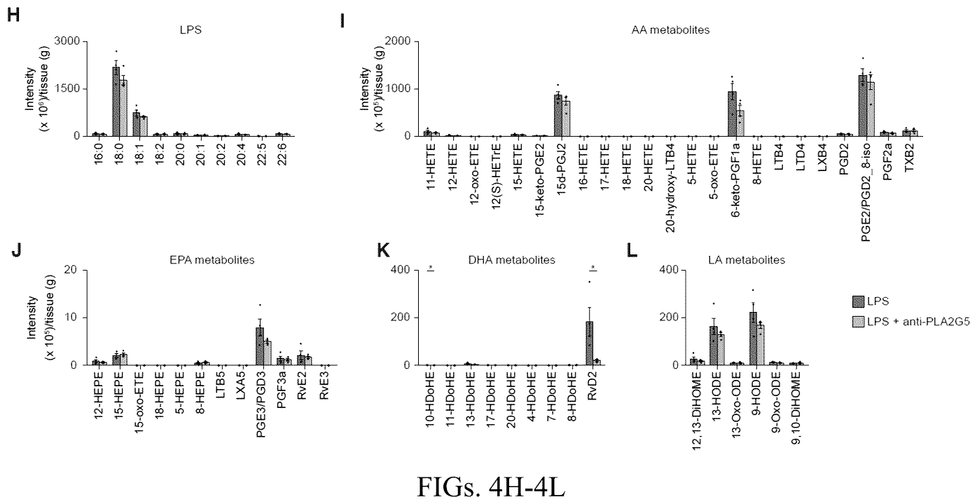

The inventors asked if PLA2G5 had detrimental effects for the host during sepsis by modulating inflammatory cytokines or lipid metabolites. A common trigger for tissue injury in sepsis is a systemic, uncontrolled inflammatory response mediated by cytokines^{4,31,32}. To test if the detrimental effects of PLA2G5 were due to the inflammatory cytokine response, the inventors measured the serum levels of TNF, IL-6, IL-12, IL-18, and IL-10 during LPS sepsis and found no difference between PLA2G5-deficient and wild-type mice (FIG. 4A). These results suggested that the negative effects of PLA2G5 on the host during sepsis, including multi-tissue damage, were independent of cytokines. Next, the inventors hypothesized that PLA2G5 could generate lipid mediators responsible for the negative impact of the secreted phospholipase on the host during sepsis. Using lipidomics on colon tissues, the inventors found that the levels of all the lipid metabolites tested showed little to no changes upon PLA2G5 blockade except for resolvin D2 (RvD2), which was downregulated in LPS-treated mice upon PLA2G5 blockade (FIG. 4B-L). Therefore, the role of PLA2G5 in sepsis appeared to be independent of systemic cytokine dysregulation or the lipolytic activities of the enzyme in colon tissue where it is induced in sepsis.

### E. Example 5: PLA2G5 Blockade Increases Splenic Red Pulp Macrophages and Iron Homeostasis

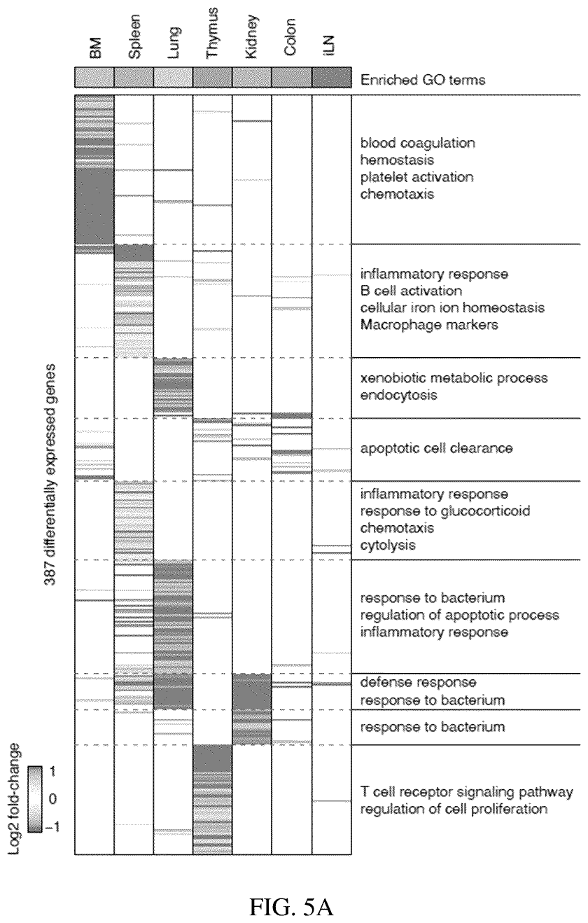

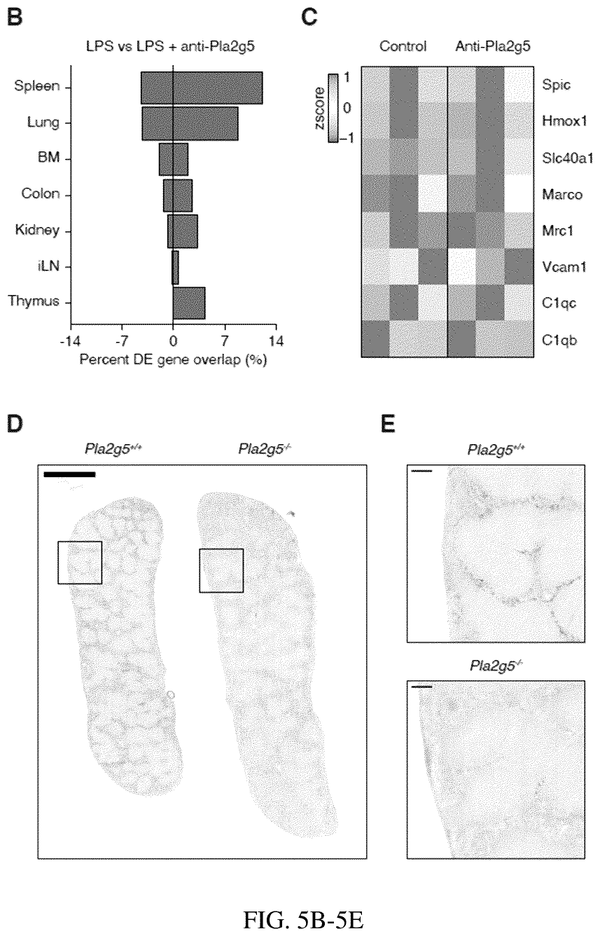

To investigate the mechanisms underlying the net, harmful effects of PLA2G5 during sepsis, the inventors measured changes in whole-tissue gene expression during sepsis across 12 organs (bone marrow, brain, colon, heart, inguinal lymph node, kidney liver, lung, skin, small intestine, spleen, and thymus) from mice treated with anti-PLA2G5 neutralizing antibodies (FIG. 5A and FIGS. S6A-C and Tables S1-2). PLA2G5 blockade impacted 8.7% (387/4,443) of all genes regulated at day 0.5 post-LPS across 7 out of the 12 organs profiled, and these effects on tissue states were most pronounced in the spleen, bone marrow, and lungs (FIG. 5A-B). Notably, by performing pathway enrichment analysis on these whole tissue expression profiles, the inventors observed a link between PLA2G5 blockade and changes in pathways related to splenic macrophages and iron homeostasis (FIG. 5A and Table S3). Since it is well-established that splenic red pulp macrophages play a role in red blood cell recycling and iron homeostasis³³, it prompted the inventors to explore the potential effects of PLA2G5 on these processes. By mining the whole-tissue gene expression profiles, the inventors found that several genes linked to splenic red pulp macrophages, such as Spic, Hmox1, and Vcam1³⁴, were upregulated upon PLA2G5 blockade in LPS-injected mice (FIG. 5C), suggesting that PLA2G5 impacts the abundance of these splenic cells. Furthermore, the increase in splenic red pulp macrophages during sepsis upon PLA2G5 blockade was associated with a decrease in Prussian blue staining of ferric iron in the spleen (FIG. 5D-E).

### F. Example 6: PLA2G5 Leads to Hemolysis by Hydrolyzing Membrane Phospholipids

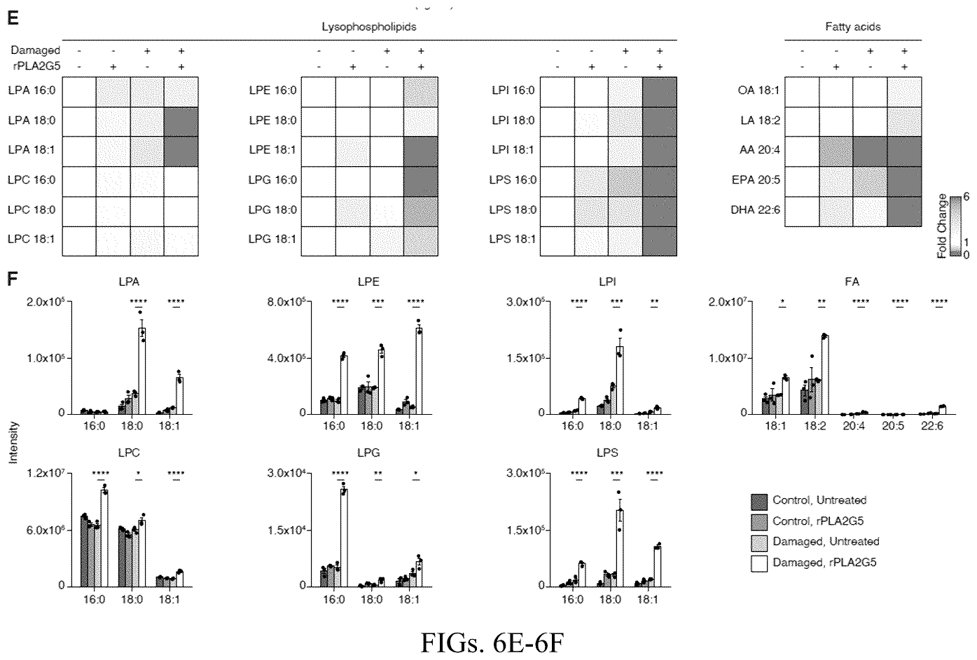

The inventors hypothesized that PLA2G5 impacted red blood cells because of the impact of PLA2G5 blockade on splenic red pulp macrophages and iron stores. To test this hypothesis, the inventors first asked if PLA2G5 could directly lyse red blood cells (RBCs) using an in vitro assay whereby recombinant PLA2G5 protein (rPLA2G5) was mixed with mouse RBCs. rPLA2G5 led to increased RBC lysis in a dose-dependent manner in vitro upon treatment with Ca²⁺ and a calcium ionophore, which damages the plasma membrane of RBCs by disrupting the distribution of phospholipids (FIG. 6A-B). Furthermore, the hemolytic effects of rPLA2G5 were dependent on its phospholipase activity as shown by adding varespladib, a broad inhibitor of secreted PLA2 enzymes, in the in vitro hemolytic assay (FIG. 6C). Next, the inventors examined the effect of osmotic pressure on the hemolysis induced by rPLA2G5. The exposure of RBCs to hypertonic conditions inhibited hemolysis mediated by rPLA2G5 while the exposure to hypotonic conditions promoted hemolysis (FIG. 6D), indicating that osmotic pressure affected the hemolytic activity of rPLA2G5.

To elucidate how PLA2G5 affected RBC membranes at the molecular level during hemolysis, the inventors performed lipidomics on erythrocytes in the presence or absence of rPLA2G5 in vitro. The inventors found that rPLA2G5 led to the release of lysophospholipids with saturation and lower degrees of unsaturation and fatty acids from RBC membranes in the in vitro hemolysis assay (FIG. 6E-F). Taken together, the data suggested that PLA2G5 hydrolyzed several phospholipids of damaged erythrocyte membranes, which led to hemolysis through osmotic pressure changes in RBCs.

### G. Example 7: PLA2G5 Acts as a Self-Venom During Sepsis by Mediating Intravascular Hemolysis

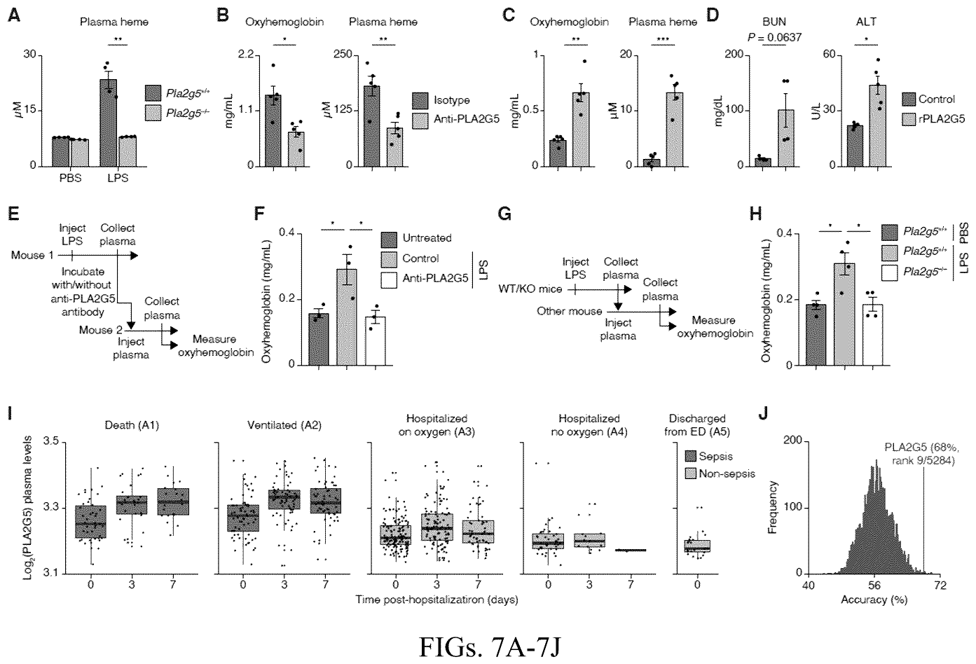

Next, the inventors hypothesized that PLA2G5 impacted red blood cells in vivo during sepsis. To test this hypothesis, the inventors measured the plasma levels of heme, a degradation product of cell-free hemoglobin, in Pla2g5^{+/+} and Pla2g5^{-/-} mice challenged with LPS. The inventors found a significant decrease in the plasma levels of heme in Pla2g5-deficient mice compared to wild type (FIG. 7A). Moreover, PLA2G5 neutralization also inhibited the elevation of the plasma levels of oxyhemoglobin and heme in mice injected with lethal dose of LPS (FIG. 7B). The inventors next asked if PLA2G5 would be sufficient to induce intravascular hemolysis in vivo by injecting rPLA2G5 in naïve mice. The inventors found that the systemic injection of rPLA2G5 in mice was sufficient to elevate the plasma levels of oxyhemoglobin and heme (FIG. 7C) and the serum levels of BUN and ALT (FIG. 7D) well above their physiological range of expression.

To test if PLA2G5 was present in the blood circulation and led to intravascular hemolysis in vivo during sepsis, the inventors performed plasma transfer experiments using donor plasma from wild-type and Pla2g5^{-/-} mice treated with LPS. Donor plasma samples from wild-type mice injected with LPS were incubated with anti-PLA2G5 or isotype control antibodies ex vivo prior to plasma transfer into naive mice. Donor plasma samples from Pla2g5′ mice injected with LPS were directly injected into naive, wild-type mice. The inventors found that plasma from LPS-injected, wild-type mice led an increase in hemolysis in naive mice, whereas plasma from PLA2G5 knockout or blockade did not (FIG. 7E-H).

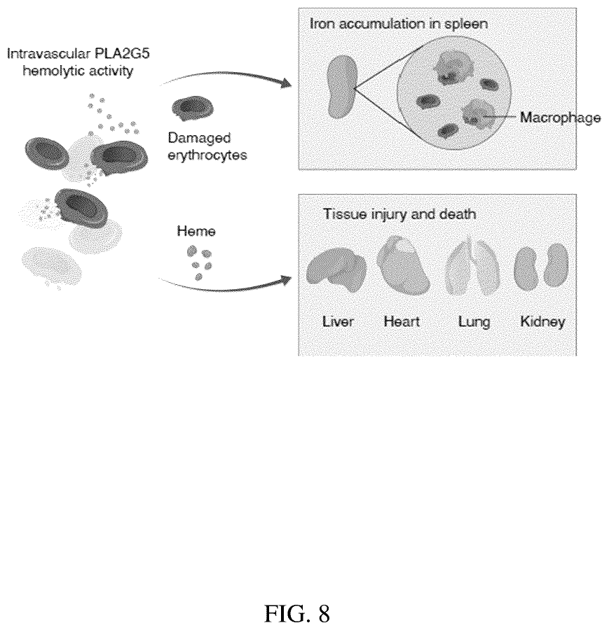

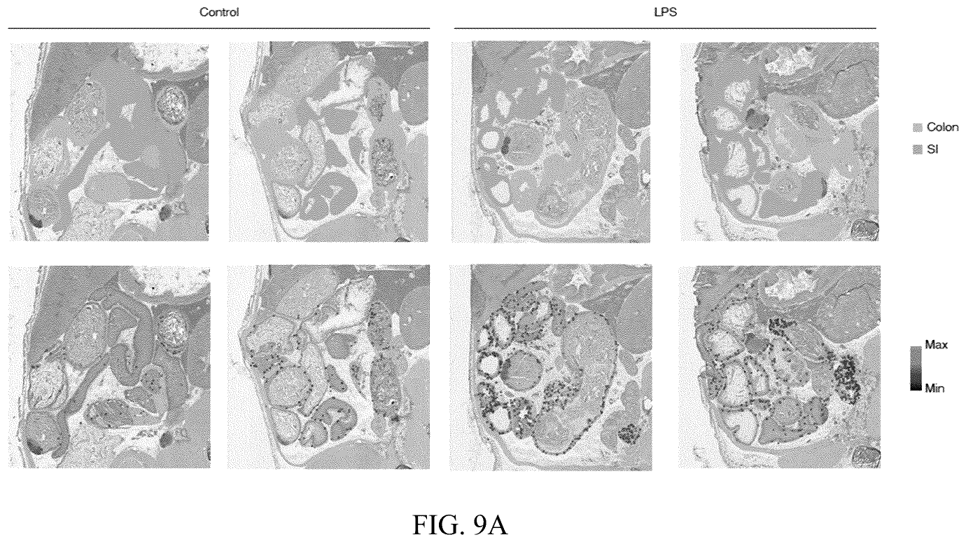

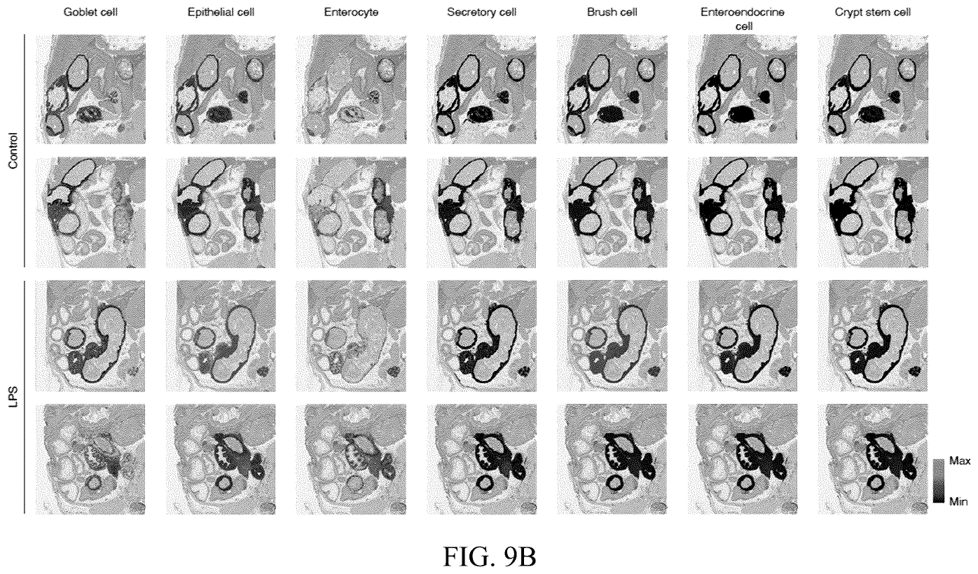

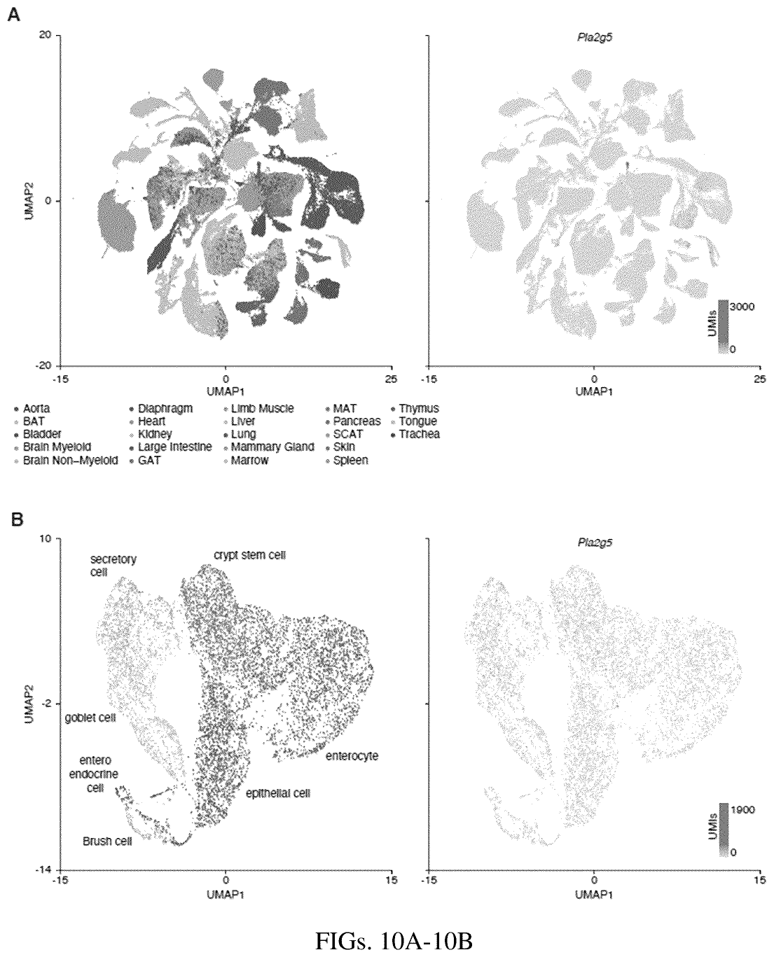

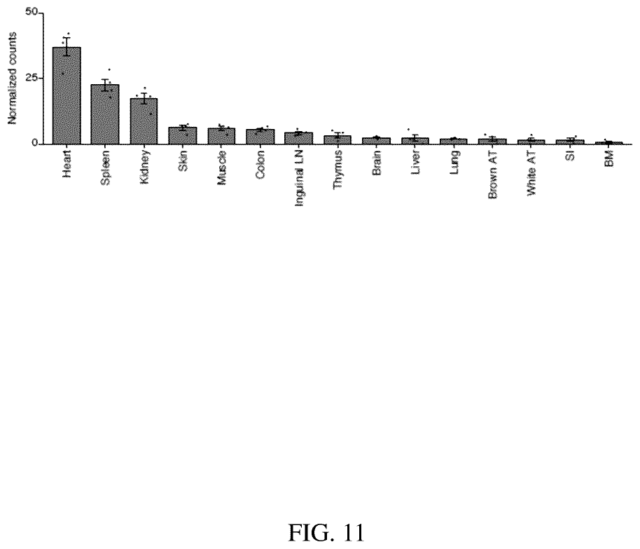

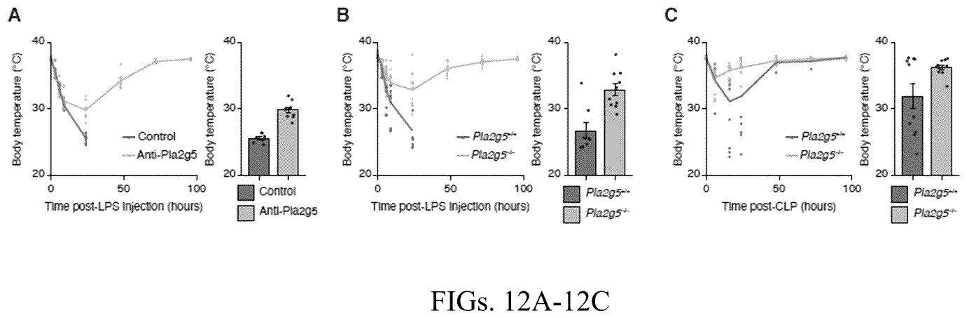

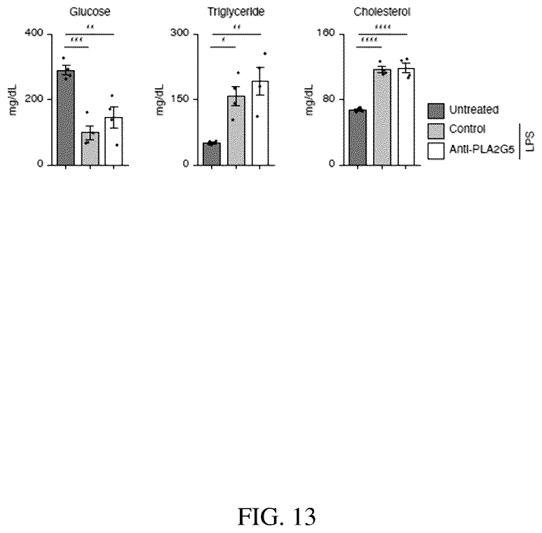

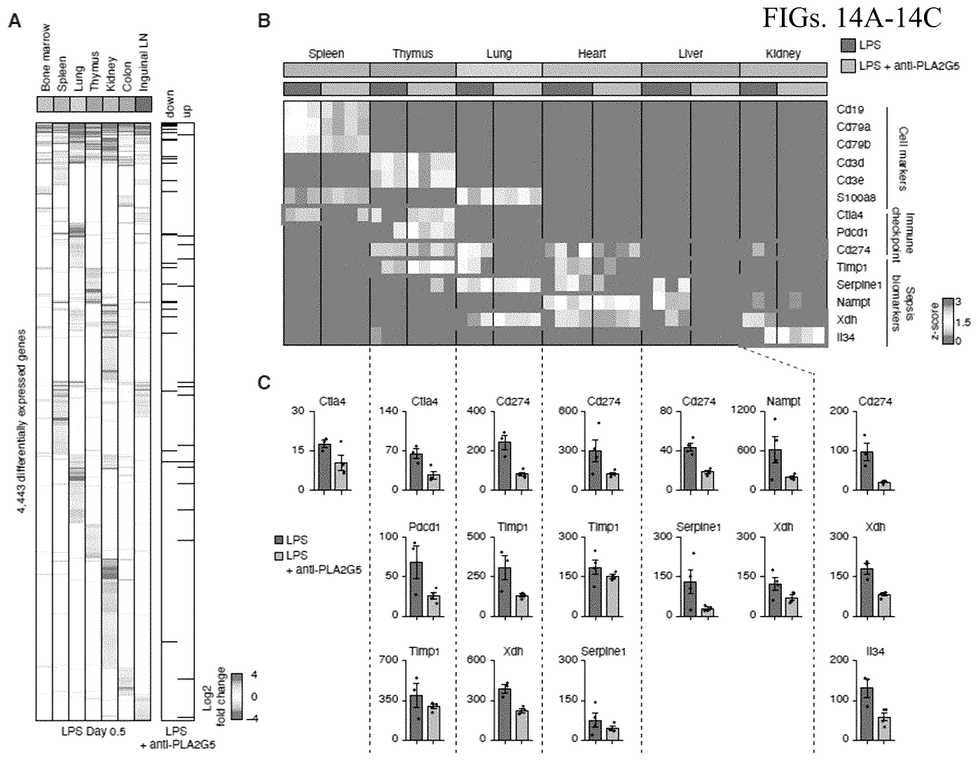

Lastly, the inventors asked if PLA2G5 could be found in the blood circulation of sepsis patients by mining publicly available plasma proteomic data from patients that match the clinical definition of sepsis¹. Using published blood proteomics data from COVID-19 patients³⁵, the inventors found that the plasma levels of PLA2G5 were significantly upregulated in patients with COVID-19-induced sepsis compared to milder, non-sepsis cases (FIG. 7I), which was predictive of patient survival with high accuracy compared to all other analytes measured in plasma (rank 9 in prediction accuracy for PLA2G5 out of a total of 5,284 proteins measured) (FIG. 7J). Overall, the results support a model whereby the release of PLA2G5 into the systemic circulation during sepsis leads to intravascular hemolysis and a concurrent increase in the blood levels of heme, a known inducer of multi-tissue injury (FIG. 8).

### H. Example 8: Discussion on Aspects Herein

The past two decades has broadened the understanding of the biology of the PLA2 family of phospholipase enzymes as both secreted and cellular PLA2 enzymes have been linked to a wide array of homeostatic and pathological processes across tissues¹⁴, prompting the search for inhibitors of these enzymes as potential therapies³⁶. Here, the inventors uncovered an interorgan axis whereby the release of the secreted PLA2G5 enzyme into the blood circulation during sepsis led to hemolysis, increased heme levels, and, subsequently, multi-organ injury with lethal consequences for the host. Pla2g5 gene expression was induced in gut cells during sepsis, pointing to the gastrointestinal tract as the likely source of bloodborne PLA2G5 in sepsis. PLA2G5 blockade decreased hemolysis and, concurrently, the burden of red blood cell death on splenic red pulp macrophages and iron homeostasis. Mechanistically, PLA2G5 directly lysed red blood cells through its lipolytic activity. The results suggest that PLA2G5 acts as self-venom with life-threatening consequences during sepsis by lysing host red blood cells in the blood circulation. Moreover, the inventors found that the plasma levels of PLA2G5 were increased in human sepsis, suggesting that this enzyme is a candidate therapeutic target to decrease intravascular hemolysis in sepsis.

Another member of the secreted PLA2 family, PLA2G2A or sPLA2-IIA, is a sepsis biomarker which is thought to release arachidonic acid, the precursor of the eicosanoid biosynthetic pathways yielding pro- and anti-inflammatory lipid species^{14,37,38}. Although PLA2G2A has been reported to hydrolyze phospholipids in vitro in erythrocyte-derived microvesicles³⁹ or activated or damaged erythrocyte membranes directly⁴⁰, the ability of PLA2G5 to hydrolyze cell membranes has been shown to be greater than that of PLA2G2A⁴¹. Furthermore, intravascular hemolysis in sepsis has been shown to be independent of PLA2G2A in clinical studies⁴².

It has been shown that PLA2G5 can act on the membranes of endothelial cells¹⁹ and bacteria^{14,16} Thus, the expression of Pla2g5 in mouse gut cells at steady-state might be indicative of a physiological role for PLA2G5 as a modulator of the microbiota, as shown for PLA2G2A⁴³, although future work is needed to test this hypothesis. Interestingly, it has been reported that PLA2G5 is also expressed in cells of the human gastrointestinal tract, including in the stomach and colon lining^{44,45}. However, it remains unknown whether PLA2G5 is induced in gut cells during human sepsis as observed in the mouse data.

The hemolytic activity of PLA2G5 in sepsis is reminiscent of the toxicity of sPLA₂ present in snake venom^{46,47}. The increase in heme blood levels due to the hemolytic activity of PLA2G5 in septic blood and downstream multi-tissue injury is consistent with previous work on the toxicity of heme on tissues during sepsis^{11,48}. Although the work demonstrated that the dominant effect of PLA2G5 during sepsis is intravascular hemolysis, further work is needed to clarify if PLA2G5 might exert any other role across various tissues in sepsis. For example, the functional consequences, if any, of the observed decrease in PLA2G5 mRNA levels in heart and spleen during sepsis remain to be investigated. Taken together, the data suggest that sepsis corrupts PLA2G5—perhaps through the disruption of gut epithelia^{49,50}—into becoming a self-venom lethal for the host.

### L. Example 9: Methods for Practicing Certain Aspects

Mice. Female C57BL/6J mice (wild-type, stock 000664) were obtained from the Jackson Laboratories. Pla2g5^{-/-} mice on a C57BL/6J genetic background were kindly provided by Steven Dudek (University of Illinois at Chicago, USA)²³. Experiments comparing Pla2g5^{+/+} to Pla2g5^{-/-} mice were performed using littermates or co-housed animals. Animals were housed in specific pathogen-free and BSL2 conditions at The University of Chicago, and all experiments were performed in accordance with the US National Institutes of Health Guide for the Care and Use of Laboratory Animals and approved by The University of Chicago Institutional Animal Care and Use Committee.

Sepsis induction. For LPS endotoxemia, mice were injected intraperitoneally with either lethal (10-15 mg/kg) or sublethal (3-5 mg/kg) doses of LPS derived from Escherichia coli 055:B5 (Sigma-Aldrich) diluted in PBS. Dosing was established for each lot of LPS by in vivo titration. Cecal ligation and puncture (CLP) was performed as described by others²⁷. Briefly, mice were anesthetized with isoflurane. A 1- to 2-cm midline laparotomy was performed, and the cecum was exposed. The cecum was ligated with 6-0 silk suture (Ethicon) and perforated as follows to vary disease severity: (1) mild sepsis: ligate at distal 33% position and perforate once with 21-G needle; (2) moderate sepsis: ligate at distal 40% position and perforate twice with a 19-G needle; (3) severe sepsis: ligate immediately below the ileocecal valve and perforate twice with a 19-G needle. The cecum was tucked back into the peritoneum and gently squeezed to extrude a small amount of fecal content. The peritoneal wall was closed using absorbable suture. The skin was closed with surgical staples. To resuscitate animals, 1 mL of saline was injected subcutaneously. Mice were temporarily placed on a heating pad for recovery. Sham operated mice underwent the same procedure except that the cecum was neither tied nor perforated.

Recombinant cytokine injections. C57BL/6J mice were injected intravenously with 2.5 μg of recombinant TNF, IL-1β, IL-6, IL-10, IL-18, or IFN-γ used alone (6 singles) or in pairwise combinations (15 pairs).

Neutralizing antibody and drug treatment. For neutralizing antibody, C57BL/6J mice were injected intraperitoneally with 50 μg of anti-PLA2G5 (clone MCL-3G1, Cayman Chemical) neutralizing antibody, or mouse IgG1 isotype control (Clone MOPC-21, BioXCell BE0083) in 100 μl of PBS 1 hour prior to LPS injection. For dexamethasone treatment, mice were injected intraperitoneally with 7 mg/kg of dexamethasone diluted in 100 μl of PBS 1 hour prior to LPS injection.

Recombinant PLA2G5 injections. C57BL/6J mice were injected intravenously with 10 μg of recombinant PLA2G5 (Cayman chemical, 10009563).

Blood analysis. Mouse whole blood was harvested by cardiac puncture and plasma and serum were isolated using lithium heparin coated Microtainer blood collection tubes (BD 365965) and Microtainer blood collection tubes (BD 365978), respectively. For flow cytometric, bead-based immunoassays, serum was diluted and processed using the LEGENDplex Mouse Macrophage/Microglia Panel (BioLegend 740846) kit. Data were acquired on the NovoCyte flow cytometer (Acea Biosciences/Agilent) and analyzed using the LEGNEDplex software v8 (BioLegend). To measure tissue injury marker levels in sera or plasma, samples were processed by In Vivo Animal Core, University of Michigan, with the following kits for BUN (BioAssay Systems DIUR-100), ALT (Cayman Chemical 700260), and troponin-I (Life Diagnostics CTNI-1-HS) levels according to the manufacturer's instructions, or with a Vet Axcel blood chemistry analyser (Alfa Wasserman). Total plasma heme was measured with the 3,3′,5,5′ tetramethylbenzidine (TMB) peroxidase assay and oxyhemoglobin with a Nanodrop One (Thermoscientific) instrument.

Tissue harvest. Tissues were harvested, frozen and stored as previously described^{51,52}. Mice were anesthetized with 2,2,2-tribromoethanol (250-500 mg/kg) and perfused transcardially with PBS containing 10 mM EDTA (to avoid signal contamination from blood in tissues). Prior to perfusion, blood was collected by cardiac puncture and stored on ice, and immediately after perfusion, tissues were placed in RNA-preserving solution (5.3 M ammonium sulfate, 25 mM sodium citrate, 20 mM EDTA) and kept at 4° C. overnight prior to transfer at −80° C. for storage. For each mouse, the inventors harvested up to 12 tissues in total: lymph node (inguinal), flank skin, thymus, heart, lung, spleen, kidney, small intestine, colon, liver, brain, and bone marrow (BM). Small intestine and colon were cut longitudinally and washed extensively in PBS to completely remove feces contamination. Bone marrow cells were collected from femora and tibiae, stored overnight in RNA-preserving solution at 4° C., centrifuged at 5,000 g for 5 min at 4° C., and cell pellets were stored at −80° C.

Whole-tissue RNA extraction. Whole-tissue RNA extraction was performed as described previously⁵². Briefly, tissues stored in RNA-preserving solution were thawed and transferred to 2 mL tubes containing 700-1500 μL (depending on tissue) of PureZOL (Bio-Rad, 7326890) or homemade Trizol-like solution (38% phenol, 0.8 M guanidine thiocyanate, 0.4 M ammonium thiocyanate, 0.1 M sodium acetate, 5% glycerol). Tissues were lysed by adding 2.8-mm ceramic beads (OMNI International, 19-646) and running 1-3 cycles of 5-45 s at 3500 rpm on the PowerLyzer 24 (QIAGEN). For liver, brain, and small intestine samples, tissues were lysed with 3-5 mL using M tubes (Miltenyi biotec, 130-096-335) and running 1-4 cycles of the RNA_02.01 program on the gentleMACS Octo Dissociator (Miltenyi biotec). Next, lysates were processed in deep 96-well plates (USA Scientific 1896-2000) by adding chloroform for phase separation by centrifugation, followed by precipitation of total RNA in the aqueous phase using magnetic beads coated with silane (Dynabeads MyOne Silane; TermoFisher Scientific 37002D), buffer RLT (QIAGEN 79216), and ethanol. Genomic DNA contamination was removed by on-bead DNase I (ThermoFisher Scientific AM2239) treatment at 37° C. for 20 min. After washing steps with 80% ethanol, RNA was eluted from beads. This RNA extraction protocol was performed on the Bravo Automated Liquid Handling Platform (Agilent)⁵². Sample concentrations were measured using a Nanodrop One (Thermo Scientific). RNA quality was confirmed using a Tapestation 4200 (Agilent Technologies).

Whole-tissue RNA sequencing. For each tissue sample, full-length cDNA was synthesized in 20 μl final reaction volume containing the following: (1) 10 μl of 10 ng/μl RNA; (2) 1 μl containing 2 pmoles of a custom RT primer biotinylated in 5′ and containing sequences from 5′ to 3′ for the Illumina read 1 primer, a 6-bp sample barcode (up to 384), a 10-bp unique molecular identifier (UMI), and an anchored oligo(dT)₃₀ for priming⁵³; and (3) 9 μl of RT mix containing 4 μl of 5×RT buffer, 1 μl of 10 mM dNTPs, 2 pmoles of template switching oligo (TSO), and 0.25 μl of Maxima H Minus Reverse Trascriptase (Thermo Scientific, EP0753). First, barcoded RT primers were added to RNA, which were then denatured at 72° C. for 1 min and snap cooled on ice. Second, the RT mix was added, and plates were incubated at 42° C. for 120 min. For each library, double stranded cDNA from up to 384 samples were pooled using DNA Clean & Concentrator-5 columns (Zymo Research, D4013), and residual RT primers were removed using exonuclease I (New England Biolabs, M0293). Full-length cDNAs were amplified with 5 to 8 cycles of single-primer PCR using the Advantage 2 PCR Kit (clontech 639206) and cleaned up using SPRIselect magnetic beads (Beckman Coulter B23318). cDNA was quantified with a Qubit dsDNA High Sensitivity Assay Kit (ThermoFisher Scientific 32851) and 50 ng of cDNA per pool of samples was tagmented using the Tagment DNA Enzyme I (Illumina 20034197) and amplified using the NEBNext Ultra II Q5 Master Mix (NEW ENGLAND BioLabs M0544L). Libraries were gel purified using 2% E-Gel EX Agarose Gels (ThermoFisher Scientific G402002), quantified with a Qubit dsDNA High Sensitivity Assay Kit (ThermoFisher Scientific Q32851) and a Tapestation 4200 (Agilent Technologies), and sequenced on the NextSeq 550 platform (Illumina).

Sequencing read files were processed to generate UMI (unique molecular identifier)⁵⁴ count matrices using the python toolkit from the bcbio-nextgen project version 1.1.5 (https://bcbio-nextgen.readthedocs.io/en/latest/)⁵⁵. In brief, reads were aligned to the mouse mm10 transcriptome with RapMap⁵⁶. Quality control metrics were compiled with a combination of FastQC (http:bioinformatics.babraham.ac.uk/projects/fastqc/), Qualimap, MultiQC (https://github.com/ewels/MultiQC)^{57,58}. Samples were demultiplexed using barcodes stored in Read 1 (first 6 bases) and raw UMI count matrices were computed using UMIs stored in Read 1 (bases 7 to 16) (https://github.com/vals/umis). Differential expression (DE) analysis was done using custom scripts in R (http://www.R-project.org). Raw count matrices were filtered to keep genes with at least 20 counts per million (cpm) or 5 UMIs in 2 samples and normalized across samples using the calcNormFactor function in edgeR⁵⁹. The inventors identified genes with indicated Benjamini and Hochberg FDR adjusted p value by comparing treated tissues and matching control tissues using limma⁶⁰. Pathway enrichment analysis was done on differentially expressed genes from indicated k-means clusters using DAVID^{61,62}. Heatmaps for RNA-seq data display the indicated numbers of transcripts and color intensities are determined by log 2 fold change value for each heatmap. The rows of each heatmap were ordered by k-means clustering of log 2 fold change values in R. All heatmaps were generated using ComplexHeatmap (https://github.com/jokergoo/ComplexHeatmap) and circlize (https://github.com/jokergoo/circlize) packages in R^{63,64}.

Histology. Tissue processing, embedding, sectioning, and immunohistochemistry using H&E and Periodic Acid Schiff were performed by the Human Tissue Resource Center at the University of Chicago. To stain ferric iron in tissue sections, spleens were frozen in OCT using dry ice, sectioned (10 μm) using a cryostat (Leica CM1850), and stained with Perl's Prussian blue (Sigma-Aldrich, P3289) and neutral red (Sigma-Aldrich, 72210). Section images were obtained using the Slideview VS200 Research Slide Scanner (Olympus).

In vitro erythrocyte lysis assay. Mouse whole blood was spun at 1,000 g, the plasma and buffy coat were removed, and red blood cells were washed three times in Hepes-buffered saline. Subsequently, a portion of erythrocytes was incubated with 100 μM CaCl₂) in Hepes-buffered saline for 3 min at 37° C. Calcium ionophore (Cayman Chemical, A23187) was added to a final concentration of 4 μM, and the cells were further incubated for 10 min at 37° C. to induce membrane phospholipid (PS) scrambling. After that, 2 mM EDTA was added to stop the reaction. In the presence or absence of 100 nM of varespladib, hypertonic solution (100 mM NaCl), or hypotonic solution (water-diluted Hepes-buffered saline), PS-exposing or untreated erythrocytes (10⁹ cells/mL) were incubated with 5, 50, or 500 ng/mL of human recombinant PLA2G5 (Cayman chemical, 10009563) for 1 h at 37° C. in Hepes-buffered saline with 2 mM of calcium for the activity of PLA2G5. Hemolysis was measured by determining the amount of oxyhemoglobin in the supernatant of erythrocyte suspensions after centrifugation at 4,000 g. The absorbance at 414 nm in the supernatant of erythrocyte suspensions was measured using Nanodrop (Thermoscientific) and compared to that of a hemolyzed aliquot of the same erythrocyte suspension treated with Triton X-100.

Lipidomics analysis of colon tissues. The procedures for lipidomics analysis using high-performance liquid chromatography coupled with electrospray tandem mass spectrometry (LC-ESI-MS/MS) were described previously⁶⁵. Briefly, C57BL/6J were injected intraperitoneally with 50 μg of anti-PLA2G5 neutralizing antibodies in 100 μl of PBS 1 hour prior to LPS injection. Twelve hours after LPS injection, mice were anesthetized with 2,2,2-tribromoethanol (250-500 mg/kg) and perfused transcardially with PBS containing 10 mM EDTA. Immediately after perfusion, colon tissues were extensively washed in PBS and frozen by liquid nitrogen and kept at −80° C. until the following procedures. Tissues were mechanically homogenized with the Precellys 24 homogenizer (Bertin Technologies, Montigny-le-Bretonneux, France) in methanol containing internal standards (500 pmol/sample of d4-labeled EPA, d5-labeled PGE2, LPC with a 17:0 fatty acyl chain (LPC17:0), and PC with two 14:0 fatty acyl chains (PE14:0-14:0)) and then incubated overnight at −20° C. For extraction of phospholipids and lysophospholipids, one-tenth of tissue lysates were added to 10 volumes of 20 mM Tris-HCl (pH 7.4) and were extracted using the method of Bligh and Dyer⁶⁶. For extraction of oxygenated fatty acid metabolites, nine-tenths of the tissue lysates were added to water (final methanol concentration of 10% (v/v)), and the lipids were extracted using an Oasis HLB cartridge (Waters, Milford, MA, USA). The samples were applied to a Kinetex C18 column (Kinetex C18, 2.1×150 mm, 1.7 μm particle; Phenomenex, Inc., Torrance, CA, USA) connected with ESI-MS/MS on a liquid chromatography (NexeraX2 system; Shimadzu Co., Kyoto, Japan) coupled with a 4000Q-TRAP quadrupole-linear ion trap hybrid mass spectrometer (AB Sciex, Framingham, MA, USA). For analyses of free fatty acids (FFAs), lysophospholipids (LPLs) and phospholipids, the samples were applied to the column and separated by a step gradient with mobile phase A (acetonitrile/methanol/water=1/1/1 (v/v/v) containing 5 mM phosphoric acid and 1 mM ammonium formate) and mobile phase B (2-propanol containing 5 μM phosphoric acid and 1 mM ammonium formate) at a flow rate of 0.2 mL/min at 50° C. For analyses of oxygenated fatty acid metabolites, the samples were applied to the column and separated using a step gradient including mobile phase C (water containing 0.1% acetic acid) and mobile phase D (acetonitrile/methanol=4/1 (v/v)) at a flow rate of 0.2 mL/min at 45° C. Identification of phospholipids, LPLs, FFAs, and oxygenated PUFAs (polyunsaturated fatty acids) metabolites was conducted by multiple reaction monitoring (MRM) transition, and quantification was performed based on the peak area of the MRM transition and the calibration curve obtained with an authentic standard for each compound⁶⁷.

Lipidomics analysis of erythrocytes. The procedures for lipidomics analysis using high-performance liquid chromatography coupled with electrospray tandem mass spectrometry (LC-ESI-MS/MS) were described above. Mouse whole blood was spun at 1,000 g, the plasma and buffy coat were removed, and red blood cells were washed three times in Hepes-buffered saline. Subsequently, a portion of erythrocytes was incubated with 100 μM CaCl₂) in Hepes-buffered saline for 3 min at 37° C. Calcium ionophore (Cayman Chemical, A23187) was added to a final concentration of 4 μM, and the cells were further incubated for 10 min at 37° C. to induce membrane phospholipid (PS) scrambling. After that, 2 mM EDTA was added to stop the reaction. PS-exposing or untreated erythrocytes (10⁹ cells/mL) were incubated with or without 100 ng/mL of human recombinant PLA2G5 (Cayman chemical, 10009563) for 1 h at 37° C. in Hepes-buffered saline with 2 mM of calcium for the activity of PLA2G5. The samples were kept at −80° C. until the following procedures.

Plasma transfer. Whole blood was harvested by cardiac puncture from donor Pla2g5^{+/+} or Pla2g5^{-/-} mice injected with LPS 12 hours prior to bleeding animals. Plasma was isolated using lithium heparin coated Vacutainer tubes (BD) and incubated with or without PLA2G5 neutralizing antibody for 4 hours at 4° C. Plasma treated with PLA2G5 neutralizing antibody was transferred intravenously (100 l) to recipient C57BL/6J mice. Plasma oxyhemoglobin in recipient mice was measured 16 hours after plasma transfer.

Whole-Mouse Spatial Transcriptomics. Mice injected with LPS or left untreated as control were euthanized with CO₂, frozen in a dry ice-hexane bath after removing all body hair and teeth, and stored at −80° C. until use. Frozen mice were embedded in a cryo-embedding medium and sectioned (10-μm thickness) using a Leica CM3600-XP cryomacrotome. Resulting whole-mouse sections were transferred onto custom, large-format microarrays for spatial transcriptomics (ST) (30-μm spot diameter with 36.65 μm center-to-center distance between spots). After transfer, sections were fixed in methanol, stained with hematoxylin and eosin, and imaged on an Olympus VS2000 slide scanner (20× magnification). Sections were permeabilized (1% pepsin), incubated for in-tissue reverse transcription, and treated with Proteinase K for tissue removal. Resulting full-length, single-stranded cDNAs were denatured and retrieved from the array using KOH and purified by column clean up (Zymo Research). cDNA was processed for single-primer PCR amplication followed by sequencing library construction using tagmentation (Nextera DNA Library Prep Kit) and final PCR amplification. Resulting libraries were sequenced on the NovaSeq 6000 (Illumina) and sequencing data was pre-processed using STAR/STARsolo 2.7.10a⁶⁸ (https://github.com/alexdobin/STAR/blob/master/docs/STARsolo.md) for read alignment using the GRCm39 mouse reference genome, spatial barcode demultiplexing, and unique molecular identifier (UMI) counting. Resulting ST data was normalized, processed for differential expression analysis, and visualized using custom Python 3.8.5 (http://www.python.org) scripts and existing packages, including Scanpy⁶⁹, scikit-image⁷⁰, and Seaborn^{71,72}. Cell type deconvolution for each ST spot was done using the CARD package²⁵.

Public single-cell RNA-sequencing data. To assess the expression pattern of Pla2g5 across the body, the inventors obtained single-cell RNA-seq data from the Tabula Muris Senis website (https://figshare.com/projects/Tabula_Muris_Senis/64982) and used the package TabulaMurisSenisData (github.com/fmicompbio/TabulaMurisSenisData) for data visualization^{26,73}.

Public proteomic and clinical index data. To assess the protein level of PLA2G5 in plasma from COVID-19 patients with sepsis, the inventors obtained proteomic and clinical index data from Mendeley Data (https://data.mendeley.com/datasets/nf853r8xsj/2)³⁵.

Decision-tree analysis. The inventors used the Python package SciPy package scikit-learn for decision-tree analysis⁷⁴. The inventors trained the decision-tree algorithm DecisionTreeClassifier. For cross-validation, the inventors first separated the data into training and test sets using cross_validate with cv=5, trained the decision-tree algorithm DecisionTreeClassifier, and then classified the test data set.

Statistical analysis. The statistical significance was determined using limma, Student's t test, or one-way ANOVA with Tukey-Kramer test. Survival analysis was performed using the log-rank test. P values are denoted by *P<0.05, **P<0.01, ***P<0.001, ****P<0.0001.

Data and Code Availability. The sequencing (whole-tissue and spatial RNA-seq) and mass spectrometry (lipidomics) datasets generated during this study have been respectively deposited in the Gene Expression Omnibus and National Metabolomics Data Repository under accession numbers GSExx and STxx, respectively. All scripts and preprocessed datasets are publicly available at the following repository: https://github.com/chevrierlab/xx.

### VIII. References

1. Singer, M., Deutschman, C. S., Seymour, C. W., Shankar-Hari, M., Annane, D., Bauer, M., Bellomo, R., Bernard, G. R., Chiche, J.-D., Coopersmith, C. M., et al. (2016). The Third International Consensus Definitions for Sepsis and Septic Shock (Sepsis-3). JAMA 315, 801. 10.1001/jama.2016.0287.
2. Rubio, I., Osuchowski, M. F., Shankar-Hari, M., Skirecki, T., Winkler, M. S., Lachmann, G., La Rosee, P., Monneret, G., Venet, F., Bauer, M., et al. (2019). Current gaps in sepsis immunology: new opportunities for translational research. The Lancet Infectious Diseases 19, e422-e436. 10.1016/S1473-3099(19)30567-5.
3. Vincent, J.-L., van der Poll, T., and Marshall, J. C. (2022). The End of “One Size Fits All” Sepsis Therapies: Toward an Individualized Approach. Biomedicines 10, 2260. 10.3390/biomedicines10092260.
4. Poll, T. van der, Shankar-Hari, M., and Wiersinga, W. J. (2021). The immunology of sepsis. Immunity 54, 2450-2464. 10.1016/j.immuni.2021.10.012.
5. Schaer, D. J., Buehler, P. W., Alayash, A. I., Belcher, J. D., and Vercellotti, G. M. (2013). Hemolysis and free hemoglobin revisited: exploring hemoglobin and hemin scavengers as a novel class of therapeutic proteins. Blood 121, 1276-1284. 10.1182/blood-2012-11-451229.
6. Rother, R. P., Bell, L., Hillmen, P., and Gladwin, M. T. (2005). The Clinical Sequelae of Intravascular Hemolysis and Extracellular Plasma HemoglobinA Novel Mechanism of Human Disease. JAMA 293, 1653-1662. 10.1001/jama.293.13.1653.
7. Gozzelino, R., Jeney, V., and Soares, M. P. (2010). Mechanisms of Cell Protection by Heme Oxygenase-1. Annu. Rev. Pharmacol. Toxicol. 50, 323-354. 10.1146/annurev.pharmtox.010909.105600.
8. Su, D., Roth, R. I., and Levin, J. (1999). Hemoglobin infusion augments the tumor necrosis factor response to bacterial endotoxin (lipopolysaccharide) in mice. Crit Care Med 27, 771-778. 10.1097/00003246-199904000-00034.
9. Fortes, G. B., Alves, L. S., de Oliveira, R., Dutra, F. F., Rodrigues, D., Fernandez, P. L., Souto-Padron, T., De Rosa, M. J., Kelliher, M., Golenbock, D., et al. (2012). Heme induces programmed necrosis on macrophages through autocrine TNF and ROS production. Blood 119, 2368-2375. 10.1182/blood-2011-08-375303.
10. Adamzik, M., Hamburger, T., Petrat, F., Peters, J., de Groot, H., and Hartmann, M. (2012). Free hemoglobin concentration in severe sepsis: methods of measurement and prediction of outcome. Critical Care 16, R125. 10.1186/cc 1425.
11. Larsen, R., Gozzelino, R., Jeney, V., Tokaji, L., Bozza, F. A., Japiassú, A. M., Bonaparte, D., Cavalcante, M. M., Chora, Â., Ferreira, A., et al. (2010). A Central Role for Free Heme in the Pathogenesis of Severe Sepsis. Science Translational Medicine 2, 51ra71-51ra71. 10.1126/scitranslmed.3001118.
12. Lan, P., Pan, K., Wang, S., Shi, Q., Yu, Y., Fu, Y., Chen, Y., Jiang, Y., Hua, X., Zhou, J., et al. (2018). High Serum Iron level is Associated with Increased Mortality in Patients with Sepsis. Scientific Reports 8, 11072. 10.1038/s41598-018-29353-2.
13. Effenberger-Neidnicht, K., and Hartmann, M. (2018). Mechanisms of Hemolysis During Sepsis. Inflammation 41, 1569-1581. 10.1007/s10753-018-0810-y.
14. Murakami, M., Sato, H., and Taketomi, Y. (2020). Updating Phospholipase A2 Biology. Biomolecules 10, 1457. 10.3390/biom10101457.
15. Lambeau, G., and Gelb, M. H. (2008). Biochemistry and Physiology of Mammalian Secreted Phospholipases A 2. Annu. Rev. Biochem. 77, 495-520. 10.1146/annurev.biochem.76.062405.154007.
16. Samuchiwal, S. K., and Balestrieri, B. (2019). Harmful and protective roles of group V phospholipase A2: Current perspectives and future directions. Biochimica et Biophysica Acta (BBA)—Molecular and Cell Biology of Lipids 1864, 819-826. 10.1016/j.bbalip.2018.10.001.
17. Yamaguchi, M., Samuchiwal, S. K., Quehenberger, O., Boyce, J. A., and Balestrieri, B. (2018). Macrophages regulate lung ILC2 activation via Pla2g5-dependent mechanisms. Mucosal Immunology 11, 615-626. 10.1038/mi.2017.99.
18. Sato, H., Taketomi, Y., Ushida, A., Isogai, Y., Kojima, T., Hirabayashi, T., Miki, Y., Yamamoto, K., Nishito, Y., Kobayashi, T., et al. (2014). The Adipocyte-Inducible Secreted Phospholipases PLA2G5 and PLA2G2E Play Distinct Roles in Obesity. Cell Metabolism 20, 119-132. 10.1016/j.cmet.2014.05.002.
19. Watanabe, K., Taketomi, Y., Miki, Y., Kugiyama, K., and Murakami, M. (2020). Group V secreted phospholipase A2 plays a protective role against aortic dissection. J. Biol. Chem. 295, 10092-10111. 10.1074/jbc.RA120.013753.
20. Munoz, N. M., Meliton, A. Y., Arm, J. P., Bonventre, J. V., Cho, W., and Leff, A. R. (2007). Deletion of Secretory Group V Phospholipase A2 Attenuates Cell Migration and Airway Hyperresponsiveness in Immunosensitized Mice. The Journal of Immunology 179, 4800-4807. 10.4049/jimmunol.179.7.4800.
21. Yano, T., Fujioka, D., Saito, Y., Kobayashi, T., Nakamura, T., Obata, J., Kawabata, K., Watanabe, K., Watanabe, Y., Mishina, H., et al. (2011). Group V secretory phospholipase A2 plays a pathogenic role in myocardial ischaemia-reperfusion injury. Cardiovasc Res 90, 335-343. 10.1093/cvr/cvq399.
22. Boilard, E., Lai, Y., Larabee, K., Balestrieri, B., Ghomashchi, F., Fujioka, D., Gobezie, R., Coblyn, J. S., Weinblatt, M. E., Massarotti, E. M., et al. (2010). A novel anti-inflammatory role for secretory phospholipase A2 in immune complex-mediated arthritis. EMBO Molecular Medicine 2, 172-187. 10.1002/emmm.201000072.
23. Muñoz, N. M., Meliton, A. Y., Meliton, L. N., Dudek, S. M., and Leff, A. R. (2009). Secretory group V phospholipase A2 regulates acute lung injury and neutrophilic inflammation caused by LPS in mice. American Journal of Physiology-Lung Cellular and Molecular Physiology 296, L879-L887. 10.1152/ajplung.90580.2008.
24. Lewis, A. J., Seymour, C. W., and Rosengart, M. R. (2016). Current Murine Models of Sepsis. Surg Infect (Larchmt) 17, 385-393. 10.1089/sur.2016.021.
25. Ma, Y., and Zhou, X. (2022). Spatially informed cell-type deconvolution for spatial transcriptomics. Nat Biotechnol 40, 1349-1359. 10.1038/s41587-022-01273-7.
26. Almanzar, N., Antony, J., Baghel, A. S., Bakerman, I., Bansal, I., Barres, B. A., Beachy, P. A., Berdnik, D., Bilen, B., Brownfield, D., et al. (2020). A single-cell transcriptomic atlas characterizes ageing tissues in the mouse. Nature, 1-6. 10.1038/s41586-020-2496-1.
27. Rittirsch, D., Huber-Lang, M. S., Flierl, M. A., and Ward, P. A. (2009). Immunodesign of experimental sepsis by cecal ligation and puncture. Nat Protoc 4, 31-36. 10.1038/nprot.2008.214.
28. Sjaastad, F. V., Jensen, I. J., Berton, R. R., Badovinac, V. P., and Griffith, T. S. (2020). Inducing Experimental Polymicrobial Sepsis by Cecal Ligation and Puncture. Current Protocols in Immunology 131, e110. 10.1002/cpim.110.
29. Muñoz, N. M., Kim, K. P., Han, S. K., Boetticher, E., Sperling, A. I., Sano, H., Han, K., Zhu, X., Leff, A. R., and Cho, W. (2000). Characterization of Monoclonal Antibodies Specific for 14-kDa Human Group V Secretory Phospholipase A₂ (hVPLA₂). Hybridoma 19, 171-176. 10.1089/02724570050031220.
30. Satake, Y., Diaz, B. L., Balestrieri, B., Lam, B. K., Kanaoka, Y., Grusby, M. J., and Arm, J. P. (2004). Role of Group V Phospholipase A2 in Zymosan-induced Eicosanoid Generation and Vascular Permeability Revealed by Targeted Gene Disruption. J. Biol. Chem. 279, 16488-16494. 10.1074/jbc.M313748200.
31. Hotchkiss, R. S., and Karl, I. E. (2003). The Pathophysiology and Treatment of Sepsis. New England Journal of Medicine 348, 138-150. 10.1056/NEJMra021333.
32. Fajgenbaum, D. C., and June, C. H. (2020). Cytokine Storm. New England Journal of Medicine 383, 2255-2273. 10.1056/NEJMra2026131. 33. Alam, M. Z., Devalaraja, S., and Haldar, M. (2017). The Heme Connection: Linking
Erythrocytes and Macrophage Biology. Front. Immunol. 0. 10.3389/fimmu.2017.00033.
34. Kohyama, M., Ise, W., Edelson, B. T., Wilker, P. R., Hildner, K., Mejia, C., Frazier, W. A., Murphy, T. L., and Murphy, K. M. (2009). Role for Spi-C in the development of red pulp macrophages and splenic iron homeostasis. Nature 457, 318-321. 10.1038/nature07472.
35. Filbin, M. R., Mehta, A., Schneider, A. M., Kays, K. R., Guess, J. R., Gentili, M., Fenyves, B. G., Charland, N. C., Gonye, A. L. K., Gushterova, I., et al. (2021). Longitudinal proteomic analysis of severe COVID-19 reveals survival-associated signatures, tissue-specific cell death, and cell-cell interactions. Cell Reports Medicine 2, 100287. 10.1016/j.xcrm.2021.100287.
36. Quach, N. D., Arnold, R. D., and Cummings, B. S. (2014). Secretory phospholipase A2 enzymes as pharmacological targets for treatment of disease. Biochemical Pharmacology 90, 338-348. 10.1016/j.bcp.2014.05.022.
37. Dore, E., and Boilard, E. (2019). Roles of secreted phospholipase A2 group IIA in inflammation and host defense. Biochimica et Biophysica Acta (BBA)—Molecular and Cell Biology of Lipids 1864, 789-802. 10.1016/j.bbalip.2018.08.017.
38. Dennis, E. A., and Norris, P. C. (2015). Eicosanoid storm in infection and inflammation. Nat Rev Immunol 15, 511-523. 10.1038/nri3859.
39. Duchez, A.-C., Boudreau, L. H., Naika, G. S., Bollinger, J., Belleannée, C., Cloutier, N., Laffont, B., Mendoza-Villarroel, R. E., Lévesque, T., Rollet-Labelle, E., et al. (2015). Platelet microparticles are internalized in neutrophils via the concerted activity of 12-lipoxygenase and secreted phospholipase A2-IIA. Proceedings of the National Academy of Sciences 112, E3564-E3573. 10.1073/pnas.1507905112.
40. Neidlinger, N. A., Larkin, S. K., Bhagat, A., Victorino, G. P., and Kuypers, F. A. (2006). Hydrolysis of Phosphatidylserine-exposing Red Blood Cells by Secretory Phospholipase A2 Generates Lysophosphatidic Acid and Results in Vascular Dysfunction*. Journal of Biological Chemistry 281, 775-781. 10.1074/jbc.M505790200.
41. Murakami, M., Taketomi, Y., Miki, Y., Sato, H., Hirabayashi, T., and Yamamoto, K. (2011). Recent progress in phospholipase A2 research: From cells to animals to humans. Progress in Lipid Research 50, 152-192. 10.1016/j.plipres.2010.12.001.
42. Dinkla, S., van Eijk, L. T., Fuchs, B., Schiller, J., Joosten, I., Brock, R., Pickkers, P., and Bosman, G. J. C. G. M. (2016). Inflammation-associated changes in lipid composition and the organization of the erythrocyte membrane. BBA Clinical 5, 186-192. 10.1016/j.bbacli.2016.03.007.
43. Miki, Y., Taketomi, Y., Kidoguchi, Y., Yamamoto, K., Muramatsu, K., Nishito, Y., Park, J., Hosomi, K., Mizuguchi, K., Kunisawa, J., et al. (2022). Group IIA secreted phospholipase A2 controls skin carcinogenesis and psoriasis by shaping the gut microbiota. JCI Insight 7. 10.1172/jci.insight.152611.
44. Masuda, S., Murakami, M., Ishikawa, Y., Ishii, T., and Kudo, I. (2005). Diverse cellular localizations of secretory phospholipase A2 enzymes in several human tissues. Biochimica et Biophysica Acta (BBA)—Molecular and Cell Biology of Lipids 1736, 200-210. 10.1016/j.bbalip.2005.08.014.
45. Mounier, C. M., Wendum, D., Greenspan, E., Fléjou, J.-F., Rosenberg, D. W., and Lambeau, G. (2008). Distinct expression pattern of the full set of secreted phospholipases A2 in human colorectal adenocarcinomas: sPLA2-III as a biomarker candidate. Br J Cancer 98, 587-595. 10.1038/sj.bjc.6604184.
46. Seifert, S. A., Armitage, J. O., and Sanchez, E. E. (2022). Snake Envenomation. N Engl J Med 386, 68-78. 10.1056/NEJMra2105228.
47. Arce-Bejarano, R., Lomonte, B., and Gutiérrez, J. M. (2014). Intravascular hemolysis induced by the venom of the Eastern coral snake, Micrurus fulvius, in a mouse model: Identification of directly hemolytic phospholipases A2. Toxicon 90, 26-35. 10.1016/j.toxicon.2014.07.010.
48. Larsen, R., Gouveia, Z., Soares, M., and Gozzelino, R. (2012). Heme Cytotoxicity and the Pathogenesis of Immune-Mediated Inflammatory Diseases. Frontiers in Pharmacology 3.
49. Fay, K. T., Ford, M. L., and Coopersmith, C. M. (2017). The intestinal microenvironment in sepsis. Biochimica et Biophysica Acta (BBA)—Molecular Basis of Disease 1863, 2574-2583. 10.1016/j.bbadis.2017.03.005.
50. Haussner, F., Chakraborty, S., Halbgebauer, R., and Huber-Lang, M. (2019). Challenge to the Intestinal Mucosa During Sepsis. Frontiers in Immunology 10.
51. Kadoki, M., Patil, A., Thaiss, C. C., Brooks, D. J., Pandey, S., Deep, D., Alvarez, D., von Andrian, U. H., Wagers, A. J., Nakai, K., et al. (2017). Organism-Level Analysis of Vaccination Reveals Networks of Protection across Tissues. Cell 171, 398-413.e21. 10.1016/j.cell.2017.08.024.
52. Pandey, S., Takahama, M., Gruenbaum, A., Zewde, M., Cheronis, K., and Chevrier, N. (2020). A whole-tissue RNA-seq toolkit for organism-wide studies of gene expression with PME-seq. Nat Protoc, 1-25. 10.1038/s41596-019-0291-y.
53. Soumillon, M., Cacchiarelli, D., Semrau, S., Oudenaarden, A. van, and Mikkelsen, T. S. (2014). Characterization of directed differentiation by high-throughput single-cell RNA-Seq. bioRxiv. 10.1101/003236.
54. Islam, S., Zeisel, A., Joost, S., La Manno, G., Zajac, P., Kasper, M., Lönnerberg, P., and Linnarsson, S. (2014). Quantitative single-cell RNA-seq with unique molecular identifiers. Nat Methods 11, 163-166. 10.1038/nmeth.2772.
55. Chapman, B., Kirchner, R., Pantano, L., Naumenko, S., Smet, M. D., Beltrame, L., Khotiainsteva, T., Sytchev, I., Guimera, R. V., Kern, J., et al. (2021). bcbio/bcbio-nextgen: 10.5281/zenodo.5781867.
56. Srivastava, A., Sarkar, H., Gupta, N., and Patro, R. (2016). RapMap: a rapid, sensitive and accurate tool for mapping RNA-seq reads to transcriptomes. Bioinformatics 32, i192-i200. 10.1093/bioinformatics/btw277.
57. Garcia-Alcalde, F., Okonechnikov, K., Carbonell, J., Cruz, L. M., Götz, S., Tarazona, S., Dopazo, J., Meyer, T. F., and Conesa, A. (2012). Qualimap: evaluating next-generation sequencing alignment data. Bioinformatics 28, 2678-2679. 10.1093/bioinformatics/bts503.
58. Ewels, P., Magnusson, M., Lundin, S., and Käller, M. (2016). MultiQC: summarize analysis results for multiple tools and samples in a single report. Bioinformatics 32, 3047-3048. 10.1093/bioinformatics/btw354.
59. Robinson, M. D., McCarthy, D. J., and Smyth, G. K. (2010). edgeR: a Bioconductor package for differential expression analysis of digital gene expression data. Bioinformatics 26, 139-140. 10.1093/bioinformatics/btp616.
60. Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Research 43, e47. 10.1093/nar/gkv007.
61. Huang, D. W., Sherman, B. T., and Lempicki, R. A. (2009). Bioinformatics enrichment tools: paths toward the comprehensive functional analysis of large gene lists. Nucleic Acids Research 37, 1-13. 10.1093/nar/gkn923.
62. Huang, D. W., Sherman, B. T., and Lempicki, R. A. (2009). Systematic and integrative analysis of large gene lists using DAVID bioinformatics resources. Nat Protoc 4, 44-57. 10.1038/nprot.2008.211.
63. Gu, Z., Eils, R., and Schlesner, M. (2016). Complex heatmaps reveal patterns and correlations in multidimensional genomic data. Bioinformatics 32, 2847-2849. 10.1093/bioinformatics/btw313.
64. Gu, Z., Gu, L., Eils, R., Schlesner, M., and Brors, B. (2014). circlize implements and enhances circular visualization in R. Bioinformatics 30, 2811-2812. 10.1093/bioinformatics/btu393.
65. Yamamoto, K., Miki, Y., Sato, H., Murase, R., Taketomi, Y., and Murakami, M. (2017). Chapter Five—Secreted Phospholipase A2 Specificity on Natural Membrane Phospholipids. Methods in Enzymology 583, 101-117. 10.1016/bs.mie.2016.09.007.
66. Bligh, E. G., and Dyer, W. J. (1959). A rapid method of total lipid extraction and purification. Can J Biochem Physiol 37, 911-917. 10.1139/o59-099.
67. Yamamoto, K., Miki, Y., Sato, M., Taketomi, Y., Nishito, Y., Taya, C., Muramatsu, K., Ikeda, K., Nakanishi, H., Taguchi, R., et al. (2015). The role of group IIF-secreted phospholipase A2 in epidermal homeostasis and hyperplasia. Journal of Experimental Medicine 212, 1901-1919. 10.1084/jem.20141904.
68. Kaminow, B., Yunusov, D., and Dobin, A. (2021). STARsolo: accurate, fast and versatile mapping/quantification of single-cell and single-nucleus RNA-seq data. 2021.05.05.442755. 10.1101/2021.05.05.442755.
69. Wolf, F. A., Angerer, P., and Theis, F. J. (2018). SCANPY: large-scale single-cell gene expression data analysis. Genome Biology 19, 15. 10.1186/s13059-017-1382-0.
70. Walt, S. van der, Schonberger, J. L., Nunez-Iglesias, J., Boulogne, F., Warner, J. D., Yager, N., Gouillart, E., and Yu, T. (2014). scikit-image: image processing in Python. PeerJ 2, e453. 10.7717/peerj.453.
71. Waskom, M. L. (2021). seaborn: statistical data visualization. Journal of Open Source Software 6, 3021. 10.21105/joss.03021.
72. Hunter, J. D. (2007). Matplotlib: A 2D Graphics Environment. Computing in Science & Engineering 9, 90-95. 10.1109/MCSE.2007.55.
73. TabulaMurisSenisData: Bulk and single-cell RNA-seq data from the Tabula Muris Senis project. R package version 1.4.0 (2022). https://github.com/fmicompbio/TabulaMurisSenisData.
74. Pedregosa, F., Varoquaux, G., Gramfort, A., Michel, V., Thirion, B., Grisel, O., Blondel, M., Prettenhofer, P., Weiss, R., Dubourg, V., et al. (2011). Scikit-learn: Machine Learning in Python. Journal of Machine Learning Research 12, 2825-2830.
74. Pedregosa, F., Varoquaux, G., Gramfort, A., Michel, V., Thirion, B., Grisel, O., Blondel, M., Prettenhofer, P., Weiss, R., Dubourg, V., et al. (2011). Scikit-learn: Machine Learning in Python. Journal of Machine Learning Research 12, 2825-2830.

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. The publications listed in the application, to the extent that they provide exemplary procedural or other details supplementary to those set forth herein, are specifically incorporated herein by reference.

1. A method for treating sepsis in a patient comprising administering a PLA2G5 targeting molecule to the patient.

2. The method of claim 1, wherein the patient has been determined to have intravascular hemolysis.

3. The method of claim 1, wherein the patient receives an administration of an additional therapy.

4. The method of claim 3, wherein the additional therapy comprises an antibiotic, fluids, insulin, a pain medication, oxygen, a blood transfusion, folic acid, corticosteroids, an immunoglobulin, rituximab, surgery, a stem cell transplant, a vasopressor, at least one fatty acid and/or LPA inhibitor, and any combination thereof.

5. The method of claim 3, wherein the patient receives an administration of the additional therapy concurrently with or in the same composition as the PLA2G3 targeting molecule.

6. The method of claim 3, wherein the patient receives an administration of the additional therapy after the administration of the PLA2G5 targeting molecule.

7. The method of claim 3, wherein the patient receives an administration of the additional therapy before the administration of the PLA2G5 targeting molecule.

8. The method of claim 1, wherein the PLA2G5 targeting molecule comprises an anti-PLA2G5 antibody or a PLA2G5-binding fragment thereof.

9. The method of claim 8, wherein the antibody is humanized or chimeric.

10. The method of claim 8, wherein the antibody is conjugated to a molecule.

11. The method of claim 1, wherein the PLA2G5 targeting molecule comprises a heavy chain variable region and/or a light chain variable region from a PLA2G5 antibody.

12. The method of claim 1, wherein the PLA2G5 targeting molecule comprises a CDR1, CDR2, and CDR3 from a heavy chain variable region and/or a CDR1, CDR2, and CDR3 from a light chain variable region.

13. The method of claim 1, wherein the PLA2G5 targeting molecule comprises a single chain variable fragment (scFV).

14. The method of claim 1, wherein a biological sample from the patient has been determined to be positive for one or more sepsis markers.

15.-23. (canceled)

24. A method of treating sepsis in a patient comprising administering an effective amount of at least one fatty acid and/or LPA inhibitor to the patient.

25. The method of claim 24, wherein the fatty acid comprises LPA, oleic acid (18:1), linoleic acid (18:2), 12-hydroxyeicosatetraenoic acid (12-HETE), 13-hydroxyoctadecadienoic acid (13-HODE), 9-hydroxyoctadecadienoic acid (9-HODE), 9-oxo-octadecadienoic acid (9-oxo-ODE), beraprost, oleic acid (18:1), linoleic acid (18:2), LPC, LPE, and any combination thereof.

26. The method of claim 25, wherein the LPA is LPA 16:0, LPA 18:0, LPA 18:1, and any combination thereof.

27.-34. (canceled)

35. A composition comprising a fatty acid and/or an LPA inhibitor and one or more therapeutic agents capable of treating sepsis.

36. The composition of claim 35, wherein the fatty acid comprises LPA, oleic acid (18:1), linoleic acid (18:2), 12-hydroxyeicosatetraenoic acid (12-HETE), 13-hydroxyoctadecadienoic acid (13-HODE), 9-hydroxyoctadecadienoic acid (9-HODE), 9-oxo-octadecadienoic acid (9-oxo-ODE), beraprost, oleic acid (18:1), linoleic acid (18:2), LPC, LPE, and any combination thereof.

37. The composition of claim 36, wherein the LPA is LPA 16:0, LPA 18:0, LPA 18:1, and any combination thereof.

38. The composition of claim 35, wherein the LPA inhibitor comprises PF8380 and/or Ki6425.

39. The composition of claim 35, wherein the therapeutic agent capable of treating sepsis comprises a PLA2G5-targeting molecule, an antibiotic, fluids, insulin, a pain medication, oxygen, a blood transfusion, folic acid, corticosteroids, an immunoglobulin, rituximab, a stem cell, a vasopressor, and any combination thereof.

40. (canceled)

41. A method of measuring protein levels in a patient, the method comprising measuring a PLA2G5 gene product in a biological sample from the patient, wherein the patient has, is suspected of having, or has been diagnosed with having sepsis.

42.-63. (canceled)
